(19) United States
(12) Patent Application Publication
(10) Pub. No.: US 2003/0165924 A1
(43) Pub. Date: Sep. 4, 2003

(54) Genes expressed in foam cell differentiation

(76) Inventors: Dov Shiffman, Palo Alto, CA (US); Roland Somogyi, Sydenham Ontario (CA); Richard Lawn, San Francisco, CA (US); Jeffrey J. Seilhamer, Los Altos Hills, CA (US); J. Gordon Porter, Newark, CA (US); Thomas Mikita, San Francisco, CA (US); Julie Tai, Cupertino, CA (US)

Correspondence Address: Incyte Genopmics Inc, Legal Department, 3160 Porter Drive, Palo Alto, CA, 94304, US

(21) Appl. No.: 10/240,965

(22) PCT Filed: Apr. 4, 2001
(86) PCT No.: PCT/US01/11128

(30) Foreign Application Priority Data
Apr. 5, 2000 (US) 60195106

(51) Int. Cl. C12Q 1/68; G01N 33/53; C07H 21/04; C07K 14/00

(52) U.S. Cl. 435/6; 435/7.1; 536/23.2; 530/350

(57) ABSTRACT

The present invention relates to purified polynucleotides and compositions comprising pluralities of polynucleotides that are differentially expressed during foam cell development and are associated with atherosclerosis. The present invention presents the use of the compositions as elements on a substrate, and provides methods for using the compositions and polynucleotides.

### TECHNICAL FIELD

The present invention relates to a plurality of polynucleotides which may be used in detecting genes modulated in human foam cells. In particular, the present invention provides for the use of these polynucleotides in the diagnosis of conditions, disorders, and diseases associated with atherosclerosis.

### BACKGROUND OF THE INVENTION

Atherosclerosis and the associated coronary artery disease and cerebral stroke represent the most common cause of death in industrialized nations. Although certain key risk factors have been identified, a full molecular characterization that elucidates the causes and provide care for this complex disease has not been achieved. Molecular characterization of growth and regression of atherosclerotic vascular lesions requires identification of the genes that contribute to features of the lesion including growth, stability, dissolution, rupture and, most lethally, induction of occlusive vessel thrombus.

An early step in the development of atherosclerosis is formation of the “fatty streak”. Lipoproteins, such as the cholesterol-rich low-density lipoprotein (LDL), accumulate in the extracellular space of the vascular intima, and undergo modification. Oxidation of LDL occurs most avidly in the sub-endothelial space where circulating antioxidant defenses are less effective. The degree of LDL oxidation affects its interaction with target cells. “Minimally oxidized” LDL (MM-LDL) is able to bind to LDL receptor but not to the oxidized LDL (Ox-LDL) or “scavenger” receptors that have been identified, including scavenger receptor types A and B, CD36, CD68/macrosialin and LOX-1 (Navab et al. (1994) Arterioscler Thromb Vasc Biol 16:831-842; Kodama et al. (1990) Nature 343:531-535; Acton et al. (1994) J Biol Chem 269:21003-21009; Endemann et al. (1993) J Biol Chem 268:11811-11816; Ramprasad et al. (1996) Proc Natl Acad Sci 92:14833-14838; Kataoka et al. (1999) Circulation 99:3110-3117). MM-LDL can increase the adherence and penetration of monocytes, stimulate the release of monocyte chemotactic protein 1 (MCP-1) by endothelial cells, and induce scavenger receptor A (SRA) and CD36 expression in macrophages (Cushing et al. (1990) Proc Natl Acad Sci 87:5134-5138; Yoshida et al. (1998) Arterioscler Thromb Vasc Biol 18:794-802; Steinberg (1997) J Biol Chem 272:20963-20966). SRA and the other scavenger receptors can bind Ox-LDL and enhance uptake of lipoprotein particles.

Mononuclear phagocytes enter the intima, differentiate into macrophages, and ingest modified bpids including Ox-LDL. In most cell types, cholesterol content is tightly controlled by feedback regulation of LDL receptors and biosynthetic enzymes (Brown and Goldstein (1986) Science 232:34-47). In imacrophages, however, the additional scavenger receptors lead to unregulated uptake of cholesterol (Brown and Goldstein (1983) Annu Rev Biochem 52:223-261) and accumulation of multiple intracellular lipid droplets producing “foam cell” phenotype. Cholesterol-engorged and dead macrophages contribute most of the mass of early “fatty streak” plaques and typical “advanced” lesions of diseased arteries. Numerous studies have described a variety of foam cell responses that contribute to growth and rupture of atherosclerotic vessel wall plaques. These responses include production of multiple growth factors and cytokie, which promote proliferation and adherence of neighboring cells; chemokines, which further attract circulating monocytes into the growing plaque; proteins, which cause remodeling of the extracellular matrix; and tissue factor, which can trigger thrombosis (Ross (1993) Nature 362:801-809; Quin et al. (1987) Proc Natl Acad Sci 84:2995-2998). Thus, cholesterol-loaded macrophages which occur in abundance in most stages of the atherosclerotic plaque formation contribute to inception of the atheroscerotic process and to eventual plaque rupture and occlusive thrombus.

During Ox-LDL uptake, macrophages produce cytokines and growth factors that elicit further cellular events that modulate atherogenesis such as smooth muscle cell proliferation and production of extracellular matrix. Additionally, these macrophages may activate genes involved in inflammation including inducible nitric oxide synthase. Thus, genes differentially expressed during foam cell formation may reasonably be expected to be markers of the atherosclerotic process.

The present invention provides a method of high-throughput screening using a plurality of probes and purified polynucleotides in a diagnostic context as markers of atherosclerosis and other cardiovascular disorders.

### SUMMARY OF THE INVENTION

The present invention provides a composition comprising a plurality of polynucleotides differentially expressed in foam cell development selected from SEQ ID NOs:1-276 as presented in the Sequence Listing. In one embodiment, each polynucleotide is an early marker of foam cell formation and is either unregulated, SEQ ID NOs:1-55, or downregulated, SEQ ID NOs:171-196. In a second embodiment, each polynucleotide is differentially expressed greater than 3-fold and is either upregulated, SEQ ID NOs:47-67, or downregulated, SEQ ID NOs:194-213. Further, the invention encompasses complements of the polynucleotides and immobilization of the polynucleotides on a substrate.

The invention provides a high throughput method for detecting altered expression of one or more polynucleotides in a sample. The method comprises hybridizing the polynucleotide composition with the sample, thereby forming one or more hybridization complexes; detecting the hybridization complexes; and comparing the hybridization complexes with those of a standard, wherein each difference in the size and intensity of a hybridization complex indicates altered expression of a polynucleotide in the sample. The sample can be from a subject with atherosclerosis and comparison with a standard defines early, mid, and late stages of that disease.

The invention also provides a high throughput method of screening a library of molecules or compounds to identify a ligand. The method comprises combining the polynucleotide composition with a library of molecules or compounds under conditions to allow specific binding; and detecting specific binding, thereby identifying a ligand. Libraries of molecules or compounds are selected from DNA molecules, RNA molecules, peptide nucleic acids (PNAs), mimetics, peptides, and proteins. The invention additionally provides a method for purifying a ligand, the method comprising combining a polynucleotide of the invention with a sample under conditions which allow specific binding, recovering the bound polynucleotide, and separating the polynucleotide from the ligand, thereby obtaining purified ligand.

The invention also provides a method of obtaining an extended or full length gene from a library of expressed or genomic nucleic acid sequences. The method comprises arranging individual library sequences on a substrate; hybridizing a polynucleotide selected from the Sequence Listing with the library sequences under conditions which allow specific binding; detecting hybridization between the polynucleotide and a sequence; and isolating the library sequence, thereby obtaining the extended or full length gene.

The present invention further provides a substantially purified polynucleotide selected from SEQ ID NOs:35-48, 68-80, 192,193, 214-224 as presented in the Sequence Listing. The invention also provides an expression vector containing the polynucleotide, a host cell containing the expression vector, and a method for producing a protein comprising culturing the host cell under conditions for the expression of protein and recovering the protein from the host cell culture.

The present invention further provides a protein encoded by a polynucleotide of the invention. The invention also provides a high-throughput method for screening a library of molecules or compounds to identify at least one ligand which specifically binds the protein. The method comprises combining the protein or a portion thereof with the library of molecules or compounds under conditions to allow specific binding and detecting specific binding, thereby identifying a ligand which specifically binds the protein. Libraries of molecules or compounds are selected from DNA molecules, RNA molecules, PNAs, mimetic, peptides, proteins, agonists, antagonists, antibodies or their fragments, immunoglobulins, inhibitors, drug compounds, and pharmaceutical agents. The invention further provides for using a protein to purify a ligand. The method comprises combining the protein or a portion thereof with a sample under conditions to allow specific binding, recovering the bound protein, and separating the protein from the ligand, thereby obtaining purified ligand. The invention also provides a pharmaceutical composition comprising the protein in conjunction with a pharmaceutical carrier and a purified antibody that specifically binds to the protein.

### DESCRIPTION OF THE TABLES

A portion of the disclosure of this patent document contains material which is subject to copyright protection. The copyright owner has no objection to the facsimilereproduction by anyone of the patent document or the patent disclosure, as it appears in the Patent and Trademark Office patent file or records, but otherwise reserves all copyright rights whatsoever.

The Sequence Listing is a compilation of polynucleotides obtained by sequencing clone inserts (isolates) of different cDNAs and identified by hybrid complex formation using the cDNAs as probes on a microarray. Each sequence is identified by a sequence identification number (SEQ ID NO) and by an Incyte ID number. The Incyte ID number represents the gene sequence that contains the clone insert.

Table 1 shows the differentially expressed genes associated with foam cell development identified by cluster analysis. Column 1 shows the SEQ ID NO, column 2 shows the Incyte ID number, and column 3 shows the gene annotation. Columns 4 through 10 show the normalized differential expression, and column 11 shows the cluster to which the gene was assigned.

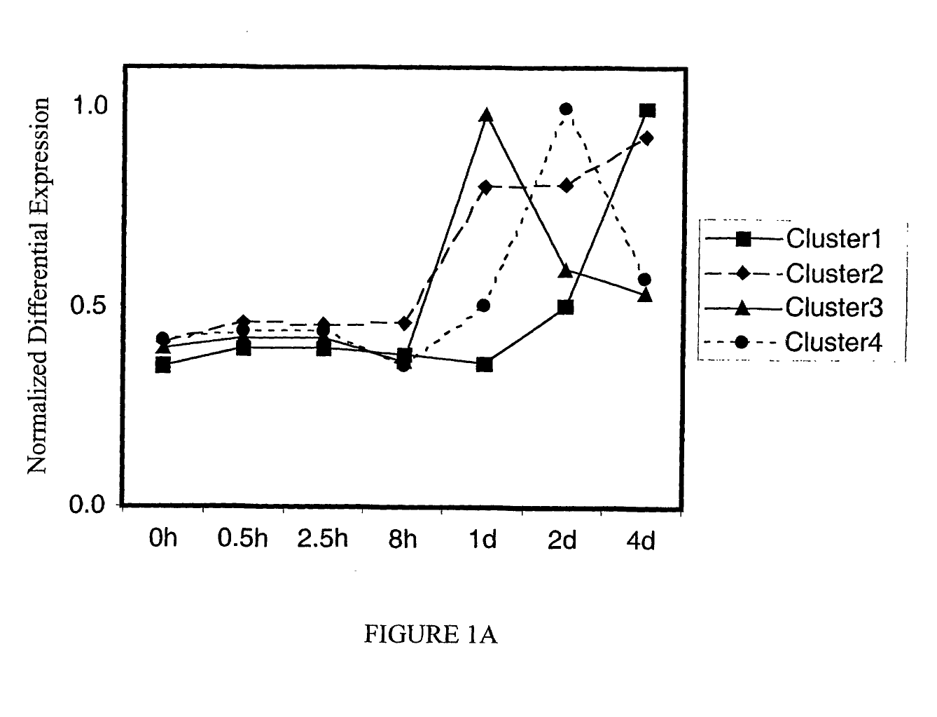

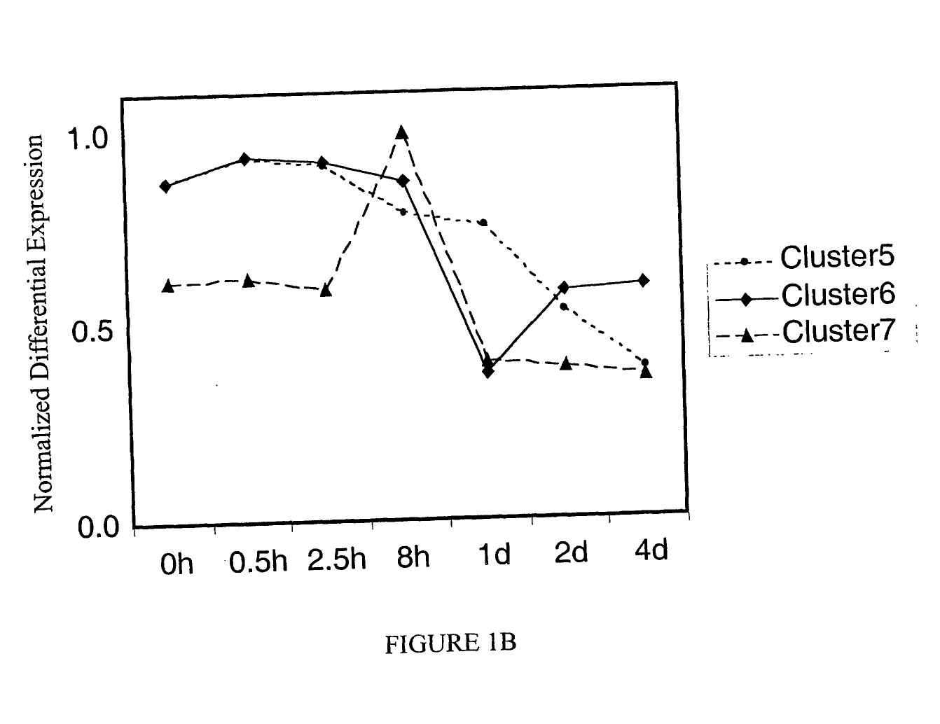

FIGS. 1A and 1B show graphs of the average normalized expression pattern over the time points for genes in each cluster. Clusters 1 through 4 contain genes which are up-regulated at days 1, 2, or 4. Clusters 5 and 6 contain genes that are down-regulated at later time points, and cluster 7 contains genes that are up-regulated at 8 hours.

Table 2 shows an identification map for each sequence. Column 1 shows the SEQ ID NO, and column 2 shows the Incyte ID number. Column 3 shows the Clone number of the Incyte clone represented on the UNIGEM V 2.0 microarray. Columns 4 and 5 show the START and STOP sites for the clone insert sequence relative to the gene sequence identified in column 2 and shown in the Sequence Listing.

Table 3 is a list of the genes that show differential expression early in foam cell differentiation. Column 1 shows the SEQ ID NO, column 2 shows the Incyte ID number, and column 3 shows the gene annotation. Columns 4 through 10 show the differential expression values for each time point. Columns 11 and 12 show the maximum change in expression up or down, respectively, over the time course. Column 12 shows the maximum difference seen over the time course.

Table 4 is a list of the genes that show greater than 3-fold differential expression during foam cell differentiation. Column 1 shows the SEQ ID NO, column 2 shows the Incyte ID number, and column 3 shows the gene annotation. Columns 4 through 10 show the differential expression values for each time point. Columns 11 and 12 show the maximum change in expression up or down, respectively, over the time course. Column 12 shows the maximum difference seen over the time course.

### DETAILED DESCRIPTION OF THE INVENTION

Before the nucleic acid sequences and methods are presented, it is to be understood that this invention is not limited to the particular machines, methods, and materials described. Although particular embodiments are described, machines, methods, and materials similar or equivalent to these embodiments may be used to practice the invention. The preferred machines, methods, and materials set forth are not intended to limit the scope of the invention which is limited only by the appended claims.

The singular forms “a”, “an”, and “the” include plural reference unless the context clearly dictates otherwise. All technical and scientific terms have the meanings commonly understood by one of ordinary skill in the art. All publications are incorporated by reference for the purpose of describing and disclosing the cell lines, vectors, and methodologies which are presented and which might be used in connection with the invention. Nothing in the specification is to be construed as an admission that the invention is not entitled to antedate such disclosure by virtue of prior invention.

Definitions

“Amplification” refers to the production of additional copies of a nucleotide sequence and is carried out using polymerase chain reaction (PCR) technologies well known in the art.

“Complementary” describes the relationship between two single-stranded nucleotide sequences that anneal by base-pairing (5′-A-G-T-3′ pairs with its complement 3′-T-C-A-5′).

“E-value” refers to the statistical probability that a match between two sequences occurred by chance.

“Derivative” refers to a polynucleotide or a polypeptide that has been subjected to a chemical modification. Illustrative of such modifications would be replacement of a hydrogen by, for example, an acetyl, acyl, alkyl, amino, formyl, or morpholino group. Derivative polynucleotides may encode polypeptides that retain the essential biological characteristics (such as catalytic and regulatory domains) of naturally occurring polypeptides.

“Fragment” refers to at least 18 consecutive nucleotides of a polynucleotide of the Sequence Listing or its complement. A “unique” fragment refers to at least 18 consecutive nucleotides of a particular polynucleotide or its complement that is specific to a polynucleotide of the Sequence Listing and that under hybridization conditions would not detect related polynucleotides in which it does not appear.

“Homology” refers to sequence similarity between a reference sequence and at least a fragment of a polynucleotide or a portion of a polypeptide.

“Hybridization complex” refers to a complex between two polynucleotides by virtue of the formation of hydrogen bonds between purines and pyrimidines.

“Immunological activity” is the capability of the natural, recombinant, or synthetic polypeptide or portion thereof to induce in an animal a specific immune response that results in the production of antibodies.

“Ligand” refers to any molecule, agent, or compound which will bind specifically to a complementary site on a polynucleotide or protein. Such ligands stabilize or modulate the activity of polynucleotides or proteins of the invention and may be composed of at least one of the following: inorganic and organic substances including nucleic acids, proteins, carbohydrates, fats, and lipids.

“Microarray” refers to an ordered arrangement of hybridizable elements on a substrate. The elements are arranged so that there are a “plurality” of elements, preferably more than one element, more preferably at least 100 elements, and even more preferably at least 1,000 elements, and most preferably at least 10,000 on a 1 cm² substrate. The maximum number of elements is unlimited, but is at least 100,000 elements. Furthermore, the hybridization signal from each of the elements is individually distinguishable. In the present and preferred embodiment, the elements comprise polynucleotide probes.

“Modulates” refers to any change in activity (increased or decreased; biological, chemical, or immunological) or lifespan resulting from specific binding between a molecule and a polynucleotide or polypeptide of the invention.

“Oligonucleotide” or “oligomer” refers to a nucleotide sequence of at least about 15 nucleotides to as many as about 60 nucleotides, preferably about 18 to 30 nucleotides, and most preferably about to 25 nucleotides that are used as a “primer” or “amplimer” in the polymerase chain reaction (PCR) or as an array element.

“Peptide nucleic acid” (PNA) refers to a DNA mimic in which nucleotide bases are attached to a pseudopeptide backbone to increase stability. PNAs, also designated antigene agents, can prevent gene expression by hybridizing to complementary messenger RNA.

“Polynucleotide” refers to an oligonucleotide, nucleotide sequence, nucleic acid molecule, DNA molecule, or any fragment or complement thereof. It may be DNA or RNA of genomic or synthetic origin, double-stranded or single-stranded, coding and/or noncoding, an exon or an intron of a genomic DNA molecule, or combined with carbohydrate, lipids, protein or inorganic elements or substances.

“portion” refers to at least six contiguous amino acids of a polypeptide encoded by a polynucleotide of the Sequence Listing. A portion may represent an amino acid sequence that is conserved among related proteins (e.g., a catalytic domain such as a kinase domain).

“Post-translational modification” of a polypeptide may involve lipidation, glycosylation, phosphorylation, acetylation, racenlization, proteolytic cleavage, and the like. These processes may occur synthetically or biochemically. Biochemical modifications will vary by cellular location, cell type, pH, enzymatic milieu, and the like.

“Probe” refers to a polynucleotide or a fragment thereof that hybridizes to a nucleic acid molecule in a sample or on a substrate. A probe is used to detect, amplify, or quantify cDNAs, endogenous genes, or transcript mRNAs by employing conventional, molecular biology techniques. As used herein, probes are the reporter molecule of hybridization reactions including Southern, northern, in situ, dot blot, array, and like technologies.

“Protein” refers to a protein or any portion thereof including a polypeptide or an oligopeptide. A portion of a polypeptide generally retains biological or immunogenic characteristics of a native protein. An “oligopeptide” is an amino acid sequence of at least about 5 residues, more preferably 10 residues and most preferably about 15 residues that are immunogenic and are used as part of a fusion protein to produce an antibody.

“Purified” refers to polynucleotides, polypeptides, antibodies, and the like, that are isolated from at least one other component with which they are naturally associated.

“Sample” is used herein in its broadest sense. A sample containing polynucleotides, polypeptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA in solution or bound to a substrate; a cell; a tissue; a tissue print; a fingerprint, skin or hair; and the like.

“Specific binding” or “specifically binding” refers to the interaction between two molecules. In the case of a polynucleotide, specific binding may involve hydrogen bonding between sense and antisense strands or between one stand and a protein which affects its replication or transcription, intercalation of a molecule or compound into the major or minor groove of the DNA molecule, or interaction with at least one molecule which functions as a transcription factor, enhancer, repressor, and the like. In the case of a polypeptide, specific binding may involve interactions with polynucleotides, as described above or with molecules or compounds such as agonists, antibodies, antagonists, and the like. Specific binding is dependent upon the presence of structural features that allow appropriate chemical or molecular interactions between molecules.

“Substrate” refers to any rigid or semi-rigid support to which molecules or compounds are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

The Invention

The present invention provides a composition comprising a plurality of polynucleotides, wherein each polynucleotide is differentially expressed in macrophages as they differentiate into foam cells. The plurality of polynucleotides comprise at least a fragment of the identified sequences, SEQ ID NOs:1-276, as presented in the Sequence Listing. Additionally, the invention provides a subset of polynucleotides whose expression is upregulated, SEQ ID NOs:1-55, or downregulated, SEQ ID NOs:171-196, early in foam cell formation. The invention also provides a subset of polynucleotides whose expression is upregulated, SEQ ID NOs:47-67, or downregulated, SEQ ID NOs:194-213, greater than 3-fold during foam cell formation. The invention also provides novel polynucleotides whose expression is upregulated, SEQ ID NOs:35-48 and 68-80, or downregulated, SEQ ID NOs:192, 193, and 214-222, during foam cell development.

Method for Selecting Polynucleotides

Human THP-1 cells (American Type Culture Collection, Manassas Va.) were grown in serum-containing medium and differentiated with 12-0-tetradecanoyl-phorbol-13-acetate (Research Biochemical International, Natick Mass.) for 24 hours. Cells were then cultured either in the presence or absence of Ox-LDL from time points ranging from 30 minutes to 4 days. Poly (A) RNA from cultured cells was prepared for expression profiling after 0, 0.5, 2.5, 8, 24, 48, and 96 hours exposure to Ox-LDL. Poly(A) RNA from experimental and control cells was labeled with separate fluorescent dyes and hybridized in time-matched pairs on UNIGEM V 2.0 arrays (Incyte Pharmaceuticals, Palo Alto Calif.).

Agglomerative cluster analysis was used to identify response patterns and to establish relationships between different gene expression profiles. Each gene measurement was normalized by dividing the expression ratios by the maximum value for each time series. The clustering process defined a hierarchical tree with the number of branches intersecting at each branch level of the tree equal to the number of clusters at that level. Division of the tree at branch level 5 divided the genes into 7 clusters of gene expression containing 276 differentially expressed genes and splice variants, SEQ ID NOs:1-276.

Table 1shows the differentially expressed genes and splice variants associated with foam cell development identified by cluster analysis. Column 1 shows the SEQ ID NO, column 2 shows the Incyte ID number, and column 3 shows the gene annotation. Columns 4 through 10 show the normalized differential expression; each gene has a maximum value of 1.0. The background shading indicates the relative expression in response to Ox-LDL; white represents relative expression ranging from 0-25% of maximum for that particular gene; light gray from 26-50%; dark gray from 51-75%; black from 76-100%. Column 11 shows the cluster to which the gene was assigned.

FIG. 1 shows a graph of the average normalized expression pattern over the time points for all the genes in each cluster. Clusters 1 through 4 contain genes which are up-regulated at days 1, 2, or 4. Clusters 5 and 6 contain genes that are down-regulated at later time points, and cluster 7 contains genes that are up-regulated at 8 hours.

Table 2 shows an ID map for each SEQ ID NO. Column 1 shows the SEQ ID NO and column 2 shows the Incyte ID number. Column 3 shows the Clone number of the Incyte clone represented on the UNIGEM V 2.0 microarray. Columns 4 and 5 show the START and STOP sites for the clone insert sequence relative to the gene sequence identified in column 2.

Table 3 is a list of the genes that show differential expression early in foam cell differentiation. Column 1 shows the SEQ ID NO, column 2 shows the Incyte ID number, and column 3 shows the gene annotation. Columns 4 through 10 show the differential expression values for each time point. Values represent treated sample divided by time matched untreated sample. Columns 11 and 12 show the maximum change in expression up or down, respectively, over the time course. Column 12 shows the maximum difference seen over the time course.

Table 4 is a list of the genes that show greater than 3-fold differential expression during foam cell differentiation. Column 1 shows the SEQ ID NO, column 2. shows the Incyte ID number, and column 3 shows the gene annotation. Columns 4 through 10 show the differential expression values for each time point. Values represent treated sample divided by time matched untreated sample. Columns 11 and 12 show the maximum change in expression up or down, respectively, over the time course. Column 12 shows the maximum difference seen over the time course.

The polynucleotides of the invention can be genomic DNA, cDNA, mRNA, or any RNA-like or DNA-like material such as peptide nucleic acids, branched DNAs and the like. Polynucleotide probes can be sense or antisense strand. Where targets are double stranded, probes may be either sense or antisense strands. Where targets are single stranded, probes are complementary single strands. In one embodiment, polynucleotides are cDNAs. In another embodiment, polynucleotides are plasmids. In the case of plasmids, the sequence of interest is the cDNA insert.

Polynucleotides can be prepared by a variety of synthetic or enzymatic methods well known in the art. Polynucleotides can be synthesized, in whole or in part, using chemical methods well known in the art (Caruthers et al. (1980) Nucleic Acids Symp. Ser. (7)215-233). Alternatively, polynucleotides can be produced enzymatically or recombinantly, by in vitro or in vivo transcription.

Nucleotide analogs can be incorporated into polynucleotide probes by methods well known in the art. The only requirement is that the incorporated nucleotide analogs of the probe must base pair with target nucleotides. For example, certain guanine nucleotides can be substituted with hypoxanthine which base pairs with cytosine residues. However, these base pairs are less stable than those between guanine and cytosine. Alternatively, adenine nucleotides can be substituted with 2,6-diaminopurine which can form stronger base pairs with thymidine than those between adenine and thymidine. Additionally, polynucleotides can include nucleotides that have been derivatized chemically or enzymatically. Typical chemical modifications include derivatization with acyl, alkyl, aryl or amino groups.

Polynucleotides can be synthesized on a substrate. Synthesis on the surface of a substrate may be accomplished using a chemical coupling procedure and a piezoelectric printing apparatus as described by Baldeschweiler et al. (PCT publication WO95/25 1116). Alternatively, the polynucleotides can be synthesized on a substrate surface using a self-addressable electronic device that controls when reagents are added as described by Heller et al. (U.S. Pat. No. 5,605,662; incorporated herein by reference).

Complementary DNA (cDNA) can be arranged and then immobilized on a substrate. Polynucleotides can be immobilized by covalent means such as by chemical bonding procedures or UV irradiation. In one such method, a cDNA is bound to a glass surface which has been modified to contain epoxide or aldehyde groups. In another case, a cDNA probe is placed on a polylysine coated surface and then UV cross-linked as described by Shalon et al. (WO95/35505). In yet another method, a DNA is actively transported from a solution to a given position on a substrate by electrical means (Heller et al., supra). Alternatively, polynucleotides, clones, plasmids or cells can be arranged on a filter. In the latter case, cells are lysed, proteins and cellular components degraded, and the DNA is coupled to the filter by UV cross-linking.

Furthermore, polynucleotides do not have to be directly bound to the substrate, but rather can be bound to the substrate through a linker group. The linker groups are typically about 6 to 50 atoms long to provide exposure of the attached probe. Preferred linker groups include ethylene glycol oligomers, diamines, diacids and the like. Reactive groups on the substrate surface react with a terminal group of the linker to bind the linker to the substrate. The other terminus of the linker is then bound to the polynucleotide.

Polynucleotides can be attached to a substrate by sequentially dispensing reagents for probe synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

Use of the Polynucleotides

The polynucleotide of the present invention may be used for a variety of purposes. For example, the composition of the invention may be used as elements on a nucroarray. The microarray can be used in high-throughput methods such as for detecting a related polynucleotide in a sample, screening libraries of molecules or compounds to identify a ligand, or diagnosing a particular cardiovascular condition, disease, or disorder such as atherosclerosis. Alternatively, a polynucleotide complementary to a given sequence of the sequence listing can inhibit or inactivate a therapeutically relevant gene related to the polynucleotide.

When the composition of the invention is employed as elements on a microarray, the polynucleotide elements are organized in an ordered fashion so that each element is present at a specified location on the substrate. Because the elements are at specified locations on the substrate, the hybridization patterns and intensities, which together create a unique expression profile, can be interpreted in terms of expression levels of particular genes and can be correlated with a particular metabolic process, condition, disorder, disease, stage of disease, or treatment.

Hybridization

The polynucleotides or fragments or complements thereof of the present invention may be used in various hybridization technologies. The polynucleotides may be naturally occurring, recombinant, or chemically synthesized; based on genomic or cDNA sequences; and labeled using a variety of reporter molecules by either PCR or enzymatic techniques. Commercial kits are available for labeling and cleanup of such polynucleotides or probes. Radioactive (Amersham Pharmacia Biotech), fluorescent (Operon Technologies, Alameda Calif.), and chemiluminescent labeling (Promega, Madison Wis.), are well known in the art. Alternatively, a polynucleotide is cloned into a commercially available vector, and probes are produced by transcription. The probe is synthesized and labeled by addition of an appropriate polymerase, such as T7 or SP6 polymerase, and at least one labeled nucleotide.

A probe may be designed or derived from unique regions of the polynucleotide, such as the 3′ untranslated region or from a conserved motif, and used in protocols to identify naturally occurring molecules encoding the same polypeptide, allelic variants, or related molecules. The probe may be DNA or RNA, is usually single stranded and should have at least 50% sequence identity to any of the nucleic acid sequences. The probe may comprise at least 18 contiguous nucleotides of a polynucleotide. Such a probe may be used under hybridization conditions that allow binding only to an identical sequence or under conditions that allow binding to a related sequence with at least one nucleotide substitution or deletion. Discovery of related sequences may also be accomplished using a pool of degenerate probes and appropriate hybridization conditions. Generally, a probe for use in Southern or northern hybridizations may be from about 400 to about 4000 nucleotides long. Such probes may be single-stranded or double-stranded and may have high binding specificity in solution-based or substrate-based hybridizations. A probe may also be an oligonucleotide that is used to detect a polynucleotide of the invention in a sample by PCR.

The stringency of hybridization is determined by G+C content of the probe, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5×saline sodium citrate (SSC) with 1% sodium dodecyl sulfate (SDS) at 60° C., that permits the formation of a hybridization complex between nucleic acid sequences that contain some mismatches. Subsequent washes are performed with buffers such as 0.2×SSC with 0.1% SDS at either 45° C. (medium stringency) or 65°-68° C. (high stringency). At high stringency, hybridization complexes will remain stable only where the polynucleotides are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or Triton X-100 (Sigma Aldrich, St. Louis Mo.) and a blocking agent such as denatured salmon sperm DNA. Selection of components and conditions for hybridization are well known to those skilled in the art and are reviewed in Ausubel (supra, pp. 6.11-6.19, 14.11-14.36, and A1-43).

Dot-blot, slot-blot, low density and high density arrays are prepared and analyzed using methods known in the art. Probes or array elements from about 18 consecutive nucleotides to about 5000 consecutive nucleotides are contemplated by the invention and used in array technologies. The preferred number of probes or array elements is at least about 40,000; a more preferred number is at least about 18,000; an even more preferred number is at least about 10,000; and a most preferred number is at least about 600 to about 800. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; PCT application WO95/11995; PCT application WO95/35505; U.S. Pat. Nos. 5,605,662; and 5,958,342.)

Screening Assays

A polynucleotide may be used to screen a library or a plurality of molecules or compounds for a ligand with specific binding affinity. The ligands may be DNA molecules, RNA molecules, PNAs, peptides, proteins such as transcription factors, enhancers, repressors, and other proteins that regulate the activity, replication, transcription, or translation of the polynucleotide in the biological system. The assay involves combining the polynucleotide or a fragment thereof with the molecules or compounds under conditions that allow specific binding and detecting the bound polynucleotide to identify at least one ligand that specifically binds the polynucleotide.

In one embodiment, the polynucleotide of the invention may be incubated with a library of isolated and purified molecules or compounds and binding activity determined by methods well known in the art, e.g., a gel-retardation assay (U.S. Pat. No. 6,010,849) or a reticulocyte lysate transcriptional assay. In another embodiment, the polynucleotide may be incubated with nuclear extracts from biopsied and/or cultured cells and tissues. Specific binding between the polynucleotide and a molecule or compound in the nuclear extract is initially determined by gel shift assay and may be later confirmed by raising antibodies against that molecule or compound. When these antibodies are added into the assay, they cause a supershift in the gel-retardation assay.

In another embodiment, the polynucleotide may be used to purify a molecule or compound using affinity chromatography methods well known in the art. In one embodiment, the polynucleotide is chemically reacted with cyanogen bromide groups on a polymeric resin or gel. Then a sample is passed over and reacts with or binds to the polynucleotide. The molecule or compound which is bound to the polynucleotide may be released from the polynucleotide by increasing the salt concentration of the flow-through medium and collected.

Purification of Ligand

The polynucleotide or a fragment thereof may be used to purify a ligand from a sample. A method for using a mammalian polynucleotide or a fragment thereof to purify a ligand would involve combining the polynucleotide or a fragment thereof with a sample under conditions to allow specific binding, recovering the bound polynucleotide, and using an appropriate agent to separate the polynucleotide from the purified ligand.

Protein Production and Uses

The polynucleotides of this application or their full length cDNAs may be used to produce purified polypeptides using recombinant DNA technologies described herein and taught in Ausubel (supra; pp. 16.1-16.62). One of the advantages of producing polypeptides by these procedures is the ability to obtain highly-enriched sources of the polypeptides thereby simplifying purification procedures. The present invention also encompasses amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.), MACVECTOR software (Genetics Computer Group, Madison Wis.) and RasMol software (www.umass.edu/microbio/rasmol) may be used to help determine which and how many amino acid residues in a particular portion of the polypeptide may be substituted, inserted, or deleted without abolishing biological or immunological activity.

Expression of Encoded Proteins

Expression of a particular cDNA may be accomplished by cloning the cDNA into an appropriate vector and transforming this vector into an appropriate host cell. The cloning vector used for the construction of the human and rat cDNA libraries may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into an appropriate host strain of E. coli. Induction of the isolated bacterial strain with isopropyltliogalactoside (IPTG) using standard methods will produce a fusion protein that contains an N terminal metbionine, the first seven residues of β-galactosidase, about 15 residues of linker, and the polypeptide encoded by the cDNA.

The cDNA may be shuttled into other vectors known to be useful for expression of protein in specific hosts. Oligonucleotides containing cloning sites and fragments of DNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized inumunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the polypeptide and the purification domain may also be used to recover the polypeptide.

Suitable expression hosts may include, but are not limited to, mammalian cells such as Chinese Hamster Ovary (CHO) and human 293 cells, insect cells such as Sf9 cells, yeast cells such as Saccharomyces cerevisiae, and bacteria such as E, coli. For each of these cell systems, a useful expression vector may also include an origin of replication and one or two selectable markers to allow selection in bacteria as well as in a transfected eukaryotic host. Vectors for use in eukaryotic expression hosts may require the addition of 3′ poly(A) tail if the polynucleotide lacks poly(A).

Additionally, the vector may contain promoters or enhancers that increase gene expression. Most promoters are host specific, and they include MMTV, SV40 or metallothionein promoters for CHO cells; trp, lac, tac or T7 promoters for bacterial hosts; or alpha factor, alcohol oxidase or PGH promoters for yeast. Adenoviral vectors with enhancers such as the rous sarcoma virus (RSV) enhancer or retroviral vectors with promoters such as the long terminal repeat (LTR) promoter may be used to drive protein expression in mammalian cell lines. Once homogeneous cultures of recombinant cells are obtained, large quantities of a secreted soluble polypeptide may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, polypeptides or portions thereof may be produced using solid-phase techniques (Stewart et al. (1969) Solid-Phase Peptide Synthesis, W H Freeman, San Francisco Calif.; Merrifield (1963) J Am Chem Soc 5:2149-2154), manually, or using machines such as the ABI 431A Peptide synthesizer (PE Biosystems, Norwalk Conn.). Polypeptides produced by any of the above methods may be used as pharmaceutical compositions to treat disorders associated with underexpression.

Screening Assays

A protein or a portion thereof encoded by the polynucleotide may be used to screen libraries or a plurality of molecules or compounds for a ligand with specific binding affinity or to purify a molecule or compound from a sample. The polypeptide or portion thereof employed in such screening may be free in solution, affixed to an abiotic or biotic substrate, or located intracellularly. For example, viable or fixed prokaryotic host cells that are stably transformed with recombinant nucleic acids that have expressed and positioned a polypeptide on their cell surface can be used in screening assays. The cells are screened against libraries or a plurality of ligands and the specificity of binding or formation of complexes between the expressed polypeptide and the ligand may be measured. The ligands may be DNA, RNA, or PNA molecules, agonists, antagonists, antibodies, immunoglobulin, inhibitors, peptides, pharmaceutical agents, proteins, drugs, or any other test molecule or compound that specifically binds the polypeptide. An exemplary assay involves combining the mammalian polypeptide or a portion thereof with the molecules or compounds under conditions that allow specific binding and detecting the bound polypeptide to identify at least one ligand that specifically binds the polypeptide.

This invention also contemplates the use of competitive drug screening assays in which neutralizing antibodies capable of binding the polypeptide specifically compete with a test compound capable of binding to the polypeptide or oligopeptide or fragment thereof. One method for high throughput screening using very small assay volumes and very small amounts of test compound is described in U.S. Pat. No. 5,876,946. Molecules or compounds identified by screening may be used in a mammalian model system to evaluate their toxicity, diagnostic, or therapeutic potential.

Purification of a Ligand

The polypeptide or a portion thereof may be used to purify a ligand from a sample. A method for using a mammalian polypeptide or a portion thereof to purify a ligand would involve combining the polypeptide or a portion thereof with a sample under conditions to allow specific binding, recovering the bound polypeptide, and using an appropriate chaotropic agent to separate the polypeptide from the purified ligand.

Production of Antibodies

A polypeptide encoded by a polynucleotide of the invention may be used to produce specific antibodies. Antibodies may be produced using an oligopeptide or a portion of the polypeptide with inherent immunological activity. Methods for producing antibodies include: 1) injecting an animal (usually goats, rabbits, or mice) with the polypeptide, or a portion or an oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobunns may be produced as taught in U.S. Pat. No. 4,816,567.

Antibodies produced using the polypeptides of the invention are useful for the diagnosis of prepathologic disorders as well as the diagnosis of chronic or acute diseases characterized by abnormalities in the expression, amount, or distribution of the polypeptide. A variety of protocols for competitive binding or immunoradiometric assays using either polyclonal or monoclonal antibodies specific for polypeptides are well known in the art. immunoassays typically involve the formation of complexes between a polypeptide and its specific binding molecule or compound and the measurement of complex formation. A two-site, monoclonal-based immunoassay utilizing monoclonal antibodies reactive to two noninterfering epitopes on a specific polypeptide is preferred, but a competitive binding assay may also be employed

Immunoassay procedures may be used to quantify expression of the polypeptide in cell cultures, in subjects with a particular disorder or in model animal systems under various conditions. Increased or decreased production of polypeptides as monitored by immunoassay may contribute to knowledge of the cellular activities associated with developmental pathways, engineered conditions or diseases, or treatment efficacy. The quantity of a given polypeptide in a given tissue may be determined by performing immunoassays on freeze-thawed detergent extracts of biological samples and comparing the slope of the binding curves to binding curves generated by purified polypeptide.

Labeling of Molecules for Assay

A wide variety of reporter molecules and conjugation techniques are known by those skilled in the art and may be used in various polynucleotide, polypeptide or antibody arrays or assays. Synthesis of labeled molecules may be achieved using Promega or Amersham Pharmacia Biotech kits for incorporation of a labeled nucleotide such as ³²p-dCTP, Cy3-dCTP or Cy5-dCTP or amino acid such as ³⁵S-methionine. Polynucleotides, polypeptides, or antibodies may be directly labeled with a reporter molecule by chemical conjugation to amines, thiols and other groups present in the molecules using reagents such as BIODIPY or FITC (Molecular Probes, Eugene Oreg.).

The polypeptides and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. Nos. 3,817,837; 3,850,752; 3,939,350; 3,996,345; 4,277,437; 4,275,149; and 4,366,241.

Diagnostics

The polynucleotides, or fragments thereof, may be used to detect and quantify altered gene expression; absence, presence, or excess expression of mRNAs; or to monitor mRNA levels during therapeutic intervention. Conditions, diseases or disorders associated with altered expression include atherosclerosis and associated complications. These polynucleotides can also be utilized as markers of treatment efficacy against the diseases noted above and other cardiovascular disorders, conditions, and diseases over a period ranging from several days to months. The diagnostic assay may use hybridization or amplification technology to compare gene expression in a biological sample from a patient to standard samples in order to detect altered gene expression. Qualitative or quantitative methods for this comparison are well known in the art.

For example, the polynucleotide may be labeled by standard methods and added to a biological sample from a patient under conditions for the formation of hybridization complexes. After an incubation period, the sample is washed and the amount of label (or signal) associated with hybridization complexes, is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established This may be accomplished by combining a biological sample taken from normal subjects, either animal or human, with a probe under conditions for hybridization or amplification Standard hybridization may be quantified by comparing the values obtained using normal subjects with values from an experiment in which a known amount of a substantially purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

Gene Expression Profiles

A gene expression profile comprises a plurality of polynucleotides and a plurality of detectable hybridization complexes, wherein each complex is formed by hybridization of one or more probes to one or more complementary sequences in a sample. The polynucleotide composition of the invention is used as elements on a microarray to analyze gene expression profiles. In one embodiment, the microarray is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the microarray is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease or treatment of the condition, disorder or disease. Novel treatment regimens may be tested in these animal models using microarrays to establish and then follow expression profiles over time. In addition, microarrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against epitopes on a protein encoded by a polynucleotide of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the protein and its specific antibody and the measurement of such complexes. These and other assays are described in Pound (supra). The method may employ a two-site, monoclonal-based immunoassay utilizing monoclonal antibodies reactive to two non-interfering epitopes, or a competitive binding assay. (See, e.g., Coligan et al. (1997) Current Protocols in Immunology, Wiley-Interscience, New York N.Y.; Pound, supra)

Therapeutics

The polynucleotides of the present invention and fragments thereof can be used in gene therapy. Polynucleotides of the invention can be delivered to a target tissue, such as mononuclear phagocytes. Expression of the protein encoded by the polynucleotide may correct a disease state associated with reduction or loss of endogenous target protein. Polynucleotides may be delivered to specific cells in vitro. Transformed cells are transferred in vivo to various tissues. Alternatively, polynucleotides may be delivered in vivo. Polynucleotides are delivered to cells or tissues using vectors such as retrovirus, adenovirus, adeno-associated virus, herpes simplex virus, and bacterial plasmids. Non-viral methods of gene delivery include cationic liposomes, polylysine conjugates, artifical viral envelopes, and direct injection of DNA (Anderson (1998) Nature 392:25-30; Dachs et al. (1997) Oncol Res 9:313-325; Chu et al. (1998) J Mol Med 76(3-4):184-192; August et al. (1997) Gene Theraov (Advances in Pharmacolog Vol. 40), Academic Press, San Diego Calif.).

In addition, expression of a particular protein can be modulated through the specific binding of an antisense polynucleotide sequence to a nucleic acid sequence which either encodes the protein or directs its expression. The antisense polynucleotide can be DNA, RNA, or nucleic acid mimics and analogs. The nucleic acid sequence can be cellular niRNA and/or genomic DNA and binding of the antisense sequence can affect translation and/or transcription, respectively. Antisense sequences can be delivered intracellularly using viral vectors or non-viral vectors as described above (Weiss et al. (1999) Cell Mol Life Sci 55(3):334-358; Agrawal (1996) Antisense Therapeutics, Humana Press Inc., Totowa N.J.).

Both polynucleotides and antisense sequences can be produced ex vivo by using any of the ABI nucleic acid synthesizers or other automated systems known in the art. Polynucleotides and antisense sequences can also be produced biologically by transforming an appropriate host cell with an expression vector containing the sequence of interest.

Molecules which modulate the expression of a polynucleotide of the invention or activity of the encoded protein are useful as therapeutics for conditions and disorders associated with an immune response. Such molecules include agonists which increase the expression or activity of the polynucleotide or encoded protein, respectively; or antagonists which decrease expression or activity of the polynucleotide or encoded protein, respectively. In one aspect, an antibody which specifically binds the protein may be used directly as an antagonist or indirectly as a targeting or delivery mechanism for bringing a pharmaceutical agent to cells or tissues which express the protein.

Additionally, any of the proteins or their ligands, or complementary nucleic acid sequences may be administered in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton Pa.).

Model Systems

Animal models may be used as bioassays where they exhibit a phenotypic response similar to that of humans and where exposure conditions are relevant to human exposures. Mannmals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

Transgenic Animal Models

Transgenic rodents that overexpress or underexpress a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types that differentiate into, for example, neural cells, hematopoietic lineages, and cardiomyocytes.

Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines that lack a functional copy of the mammalian gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition These methods have been used to model several human diseases.

As described herein, the uses of the polynucleotides, provided in the Sequence Listing of this application, and their encoded polypeptides are exemplary of known techniques and are not intended to reflect any limitation on their use in any technique that would be known to the person of average skill in the art. Furthermore, the polynucleotides provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining or assembling full length cDNA sequences that will be known to those skilled in the art.

It is to be understood that the invention is not limited to the particular methodology, protocols, and reagents described, as these may vary. It is also understood that the terminology used herein is for the purpose of describing particular embodiments only, and is not intended to limit the scope of the present invention which will be limited only by the appended claims. The examples below are provided to illustrate the subject invention and are not included for the purpose of limiting the invention.

### EXAMPLES

I. Construction of cDNA Libraries

RNA was purchased from Clontech Laboratories, Inc. (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Life Technologies, Rockville Md.). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNase. For most libraries, poly(A) RNA was isolated using oligo d(T)-coupled paramagnetic particles (Promega), OLIGOTEX latex particles (Qiagen, Valencia Calif.), or an OLIGOTEX mRNA purification kit (Qiagen). Alternatively, poly(A) RNA was isolated directly from tissue lysates using other kits, including the POLY(A)PURE mRNA purification kit (Ambion, Austin Tex.).

In some cases, Stratagene (La Jolla, Calif.) was provided with RNA and constructed the corresponding cDNA libraries. Otherwise, cDNA was synthesized and cDNA libraries were constructed with the UNIZAP vector system (Stratagene) or SUPERSCRIPT plasrid system (Life Technologies) using the recommended procedures or similar methods known in the art (See Ausubel, supra, Units 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech, Piscataway N.J.) or preparative agarose gel electrophoresis. cDNAs were ligated into compatible restriction enzyme sites of the polylinker of the PBLUESCRIPT plasmid (Stratagene), PSPORT1 plasmid (Life Technologies), or PINCY plasmid (Incyte Pharmaceuticals). Recombinant plasmids were transformed into XL1-Blue, XL1-BlueMRF, or SOLR competent E. coli cells (Stratagene) or DH5α, DH10B, or ELECTROMAX DH10B competent E, coli cells (Life Technologies).

In some cases, libraries were superinfected with a 5× excess of the helper phage, M13K07, according to the method of Vieira et al. (1987, Methods Enzymol. 153:3-11) and normalized or subtracted using a methodology adapted from Soares (1994, Proc Natl Acad Sci 91:9228-9232), Swaroop et al. (1991, Nucl Acids Res 19:1954), and Bonaldo et al. (1996, Genome Research 6:791-806). The modified Soares normalization procedure was utilized to reduce the repetitive cloning of highly expressed high abundance cDNAs while maintaining the overall sequence complexity of the library. Modification included significantly longer hybridization times which allowed for increased gene discovery rates by biasing the normalized libraries toward those infrequently expressed low-abundance cDNAs which are poorly represented in a standard transcript image (Soares et al., supra).

II. Isolation and Sequencing of cDNA Clones

Plasmids were recovered from host cells by in vivo excision using the UNIZAP vector system (Stratagene) or by cell lysis. Plasmids were purified using one of the following: the Magic or WIZARD Minipreps DNA purification system (Promega); the AGTC Miniprep purification kit Edge BioSystems, Gaithersburg Md.); the QIAWELL 8, QIAWELL 8 Plus, or QIAWELL 8 Ultra plasmid purification systems, or the R.E.A.L. PREP 96 plasmid purification kit (QIAGEN). Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao (1994) Anal Biochem 216:1-14). Host cell lysis and thermal cycling steps were carried out in a singlereaction mixture. Samples wereprocessed and stored in 384-well plates, and the concentration of amplified plasmid DNA was quantified fluorometrically using PICOGREEN dye (Molecular Probes) and a FLUOROSKAN II fluorescence scanner (absystems Oy, Helsinki, Finland).

cDNA sequencing reactions were processed using standard methods or high-throughput instrumentation such as the ABI CATALYST 800 thermal cycler (PE Biosystems) or the DNA ENGINE thermal cycler (MJ Research, Watertown Mass.) in conjunction with the HYDRA microdispenser (Robbins Scientific, Sunnyvale Calif.) or the MICROLAB 2200 system (Hamilton, Reno Nev.). cDNA sequencing reactions were prepared using reagents provided by Amersham Pharmacia Biotech or supplied in ABI sequencing kits such as the ABI PRISM BIGDYE cycle sequencing kit (PE Biosystems). Electrophoretic separation of cDNA sequencing reactions and detection of labeled polynucleotides were carried out using the MEGABACE 1000 DNA sequencing system (Amersham Pharmacia Biotech); the ABI PRISM 373 or 377 sequencing system (PE Biosystems) in conjunction with standard ABI protocols and base calling software; or other sequence analysis systems known in the art. Reading frames within the cDNA sequences were identified using standard methods (reviewed in Ausubel, supra, Unit 7.7).

III. Extension of cDNA Sequences

Nucleic acid sequences were extended using Incyte cDNA clones and oligonucleotide primers. One primer was synthesized to initiate 5′ extension of the known fragment, and the other, to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (MJ Research). The reaction mix contained DNA template, 200 nmol of each primer, reaction buffer containing Mg⁺², (NH₄)2SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B (Incyte Pharmaceuticals): Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C.. In the alternative, the parameters for primer pair T7 and SK+ (Stratagene) were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 mm; Step 7: storage at 4° C.

The concentration of DNA in each well was determined by dispensing 100 μl PICOGREEN reagent (0.25% reagent in 1×TE, v/v; Molecular Probes) and 0.5 of undiluted PCR product into each well of an opaque fluorimeter plate (Corning Costar, Acton Mass.) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleic acids were desalted and concentrated, transferred to 384-well plates, digested with CviJI cholera virus endonuclease (Molecular Biology Research, Madison Wis.), and sonicated or sheared prior to religation into pUC18 vector (Amersham Pharmacia Biotech). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC 18 vector (Amersham Pharmacia Biotech), treated with Pfu DNA polymerase (Stratagene) to fill-in restriction site overhangs, and transfected into competent E, coli cells. Transformed cells were selected on antibiotic-containing media, and individual colonies were picked and cultured overnight at 37° C. in 384-well plates in LB/2× carbenicillin liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (Amersham Pharmacia Biotech) or the ABI PRISM BIGDYE terminator cycle sequencing kit (PE Biosystems).

IV. Assembly and Analysis of Sequences

Component nucleotide sequences from chromatograms were subjected to PHRED analysis (Phil's Revised Editing Program; Phil Green, University of Washington, Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs. Sequences were screened using the BLOCK 2 program (Incyte Pharmaceuticals), a motif analysis program based on sequence information contained in the SWISS-PROT and PROSITE databases (Bairoch et al. (1997) Nucleic Acids Res. 25:217-221; Attwood et al. (1997) J. Chem. Inf. Comput. Sci. 37:417-424).

Processed sequences were subjected to assembly procedures in which the sequences were assigned to bins, one sequence per bin. Sequences in each bin were assembled to produce consensus sequences, templates. Subsequent new sequences were added to existing bins using the Basic Local Alignment Search Tool (BLAST; Altschul (1993) J. Mol. Evol. 36:290-300; Altschul et al. (1990) J. Mol. Biol. 215:403-410; Karlin et al. (1988) Proc. Natl. Acad. Sci. 85:841-845), BLASTn (v.1.4, WashU), and CROSSMATCH software (Phil Green, supra). Candidate pairs were identified as all BLAST hits having a quality score greater than or equal to 150. Alignments of at least 82% local identity were accepted into the bin. The component sequences from each bin were assembled using PHRAP (Phil's Revised Alignment Program; Phil Green, supra). Bins with several overlapping component sequences were assembled using DEEP PHRAP (Phil Green, supra).

Bins were compared against each other, and those having local similarity of at least 82% were combined and reassembled. Reassembled bins having templates of insufficient overlap (less than 95% local identity) were re-split. Assembled templates were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to generate the template sequences found in the LIFESEQ GOLD database (Incyte Pharmaceuticals).

The assembled templates were annotated using the following procedure. Template sequences were analyzed using BLASTn (v2.0, NCBI) versus GBpri (GenBank version 109). “Hits” were defined as an exact match having from 95% local identity over 200 base pairs through 100% local identity over 100 base pairs, or a homolog match having an E-value of 1×10⁻⁸. The hits were subjected to frameshift FASTx versus GENPEPT (GenBank version 109). In this analysis, a homolog match was defined as having an E-value of 1×10⁻⁸. The assembly method used above was described in “Database and System for Storing, Comparing and Displaying Related Biomolecular Sequence Information,” U.S. Ser. No. 09/276,534, filed Mar. 25, 1999, incorporated by reference herein, and the LIFESEQ GOLD user manual (Incyte Pharmaceuticals).

Following assembly, template sequences were subjected to motif, BLAST, Hidden Markov Model (HMM; Pearson and Lipman (1988) Proc Natl Acad Sci 85:2444-2448; Smith and Waterman (1981) J Mol Biol 147:195-197), and functional analyses, and categorized in protein hierarchies using methods described in “Database System Employing Protein Function Hierarchies for Viewing Biomolecular Sequence Data,” U.S. Ser. No. 08/812,290, filed Mar. 6, 1997; “Relational Database for Storing Biomolecule Information,” U.S. Ser. No. 08/947,845, filed October 9, 1997; “Project-Based Full-Length Biomolecular Sequence Database,” U.S. Pat. No. 5,953,727;; and “Relational Database and System for Storing Information Relating to Biomolecular Sequences,” U.S. Ser. No. 09/034,807, filed Mar. 4, 1998, all of which are incorporated by reference herein. Template sequences may be further queried against public databases such as the GenBank rodent, mammalian, vertebrate, eukaryote, prokaryote, and human EST databases.

V. Preparation of Microarrays

The polynucleotides present on the human UNIGEM V 2.0 microarray (Incyte Pharmaceuticals) represent template sequences derived from the LIFESEQ GOLD assembled human sequence database (incyte Pharmaceuticals) based on a non-redundant set of gene-oriented clusters derived from IMAGE (integrated molecular analysis of genomes and their expression) cDNA library clones and derived ESTs in the gbEST database (National Center for Biotechnology Information, National Library of Medicine, Bethesda, Md.). A single clone representing each particular template was used on the microarray. Polynucleotides were amplified from bacterial cells using primers complementary to vector sequences flanking the cDNA insert. Thirty cycles of PCR increased the initial quantity of polynucleotide from 1-2 ng to a final quantity greater than 5 μg. Amplified polynucleotides were then purified using SEPHACRYL400 columns (Amersham Pharmacia Biotech).

Purified polynucleotides were immobilized on polymer-coated glass slides. Glass microscope slides (Corning, Corning N.Y.) were cleaned by ultrasound in 0.1% SDS and acetone, with extensive distilled water washes between and after treatments. Glass slides were etched in 4% hydrofluoric acid (VWR Scientific Products Corporation, West Chester Pa.), washed extensively in distilled water, and coated with 0.05% aminopropyl silane (Sigma Aldrich, St. Louis Mo.) in 95% ethanol. Coated slides were cured in a 110° C. oven. polynucleotides were applied to the coated glass substrate using a procedure described in U.S. Pat. No. 5,807,522, incorporated hereinby reference. One microliter of the polynucleotide at an average concentration of 100 ng/ul was loaded into the open capillary printing element by a high-speed robotic apparatus which then deposited about 5 nl of polynucleotide per slide.

Microarrays were UV-crosslinked using a STRATALINKER UV-crosslinker (Stratagene), and then washed at room temperature once in 0.2% SDS and three times in distilled water. Non-specific binding sites were blocked by incubation of microarrays in 0.2% casein in phosphate buffered saline (Tropix, Bedford Mass.) for 30 minutes at 60° C. followed by washes in 0.2% SDS and distilled water as before.

VI. Preparation of Target Polynucleotides

Human THP-1 cells (American Type Culture Collection, Manassas Va.) were grown in RPMI1640 medium containing 10% fetal serum (v/v), 0.45% glucose (w/v), 10 mM Hepes, 1 mM sodium pyruvate, 1×10-⁻⁵ M β-mercaptoethanol, penicillin (100 units/ml) and streptomycin (100 mg/ml). For oxidized-LDL loading experiments, cells were seeded at a density of 1×10⁶ cells/il in medium containing 12-0-tetradecanoyl-phorbol-13-acetate (Research Biochemical International, Natick Mass.) at 1×10⁻⁷ M for 24 hr. The medium was then replaced by culture medium with or without 100 μg/ml of CuSO₄ “fully” oxidized LDL (Intracel, Rockville Md.) according to the method of Hammer et al. (1995; Arterio Thromb Vasc Biol 15:704-713). Medium was replaced every two days during the time of culture. Cells were treated with Ox-LDL over time points ranging from 30 minutes to 4 days. During this period, cells remained adherent and had a typical speckled Nile red staining pattern. RNA was prepared for expression profiling at 0, 0.5, 2.5, and 8 hours, and 1, 2, and 4 days of Ox-LDL exposure.

Total RNA was extracted using the RNA STAT-60 kit (Tel-Test, Friendswood Tex.). Poly(A) RNA was purified using the POLYATRACT mRNA isolation system (Promega). Each poly(A) RNA sample was reverse transcribed using MMLV reverse-transcriptase, 0.05 pg/μl oligo-dT primer (21mer), 1× first strand buffer, 0.03 units/ul RNase inhibitor, 500 uM DATP, 500 uM dGTP, 500 uM dTTP, 40 uM dCTP, and 40 uM either dCTP-Cy3 or dCTP-Cy5 (Amersham Pharmacia Biotech). The reverse transcription reaction was performed in a 25 ml volume containing 200 ng poly(A) RNA using the GEMBRIGHT kit (Incyte Pharmaceuticals). Specific control poly(A) RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, control mRNAs (YCFR06, YCFR45, YCFR67, and YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37° C. for 2 hr, treated with 2.5 ml of 0.5M sodium hydroxide, and incubated for 20 minutes at 85° C. to the stop the reaction and degrade the RNA.

Probes were purified using two successive CHROMA SPIN 30 gel filtration spin columns (Clontech). Cy3- and Cy5-labeled reaction samples were combined as described below and ethanol precipitated using 1 ml of glycogen (1 mg/ml), 60 ml sodium acetate, and 300 ml of 100% ethanol. The probe was then dried to completion using a SpeedVAC system (Savant Instruments, Holbrook N.Y.) and resuspended in 14 μl 5×SSC/0.2% SDS.

VII. Hybridization and Detection

Hybridization reactions contained 9 μl of probe mixture consisting of 0.2 μg each of Cy3 and Cy5 labeled cDNA synthesis products from pairs of matched time point experimental and control cells in 5×SSC, 0.2% SDS hybridization buffer. The target mixture was heated to 65° C. for 5 minutes and was aliquoted onto the microarray surface and covered with an 1.8 cm² coverslip. The microarrays were transferred to a waterproof chamber having a cavity just slightly larger than a microscope slide. The chamber was kept at 100% humidity internally by the addition of 140 μl of 5×SSC in a corner of the chamber. The chamber containing the microarrays was incubated for about 6.5 hours at 60° C. The microarrays were washed for 10 min at 45° C. in low stringency wash buffer (1×SSC, 0.1% SDS), three times for 10 minutes each at 45° C. in high stringency wash buffer (0.1×SSC), and dried.

Reporter-labeled hybridization complexes were detected with a microscope equipped with an Innova 70 mixed gas 10 W laser (Coherent, Santa Clara Calif.) capable of generating spectral lines at 488 nm for excitation of Cy3 and at 632 nm for excitation of CyS. The excitation laser light was focused on the microarray using a 20× microscope objective (Nikon, Melville N.Y.). The slide containing the microarray was placed on a computer-controlled X-Y stage on the microscope and raster-scanned past the objective. The 1.8 cm×1.8 cm microarray used in the present example was scanned with a resolution of 20 micrometers.

In two separate scans, the mixed gas multiline laser excited the two fluorophores sequentially. Emitted light was split, based on wavelength, into two photomultiplier tube detectors (PMT R1477; Hamamatsu Photonics Systems, Bridgewater N.J.) corresponding to the two fluorophores. Appropriate filters positioned between the microarray and the photomultiplier tubes were used to filter the signals. The emission maxima of the fluorophores used were 565 nm for Cy3 and 650 nm for Cy5. Each microarray was typically scanned twice, one scan per fluorophore using the appropriate filters at the laser source, although the apparatus was capable of recording the spectra from both fluorophores simultaneously.

The sensitivity of the scans was calibrated using the signal intensity generated by a cDNA control species. Samples of the calibrating cDNA were separately labeled with the two fluorophores and identical amounts of each were added to the hybridization mixture. A specific location on the microarray contained a complementary DNA sequence, allowing the intensity of the signal at that location to be correlated with a weight ratio of hybridizing species of 1:100,000.

The output of the photomultiplier tube was digitized using a 12-bit RTI-835H analog-to-digital (AID) conversion board (Analog Devices, Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incyte Pharmaceuticals).

VIII. Data Analysis and Results

An agglomerative cluster analysis was used to identify the typical response patterns and establish the relationships between the different gene expression profiles. Each gene measurement was first normalized by dividing the expression ratios by the maximum value for each time series. To emphasize the variation from one time point to the next, slopes were added to the expression vectors by taking the expression differences between consecutive time points. The Euclidean distance was used as a similarity measure for the expression responses.

The agglomerative algorithm employed constructs a dendrogram. Starting with N clusters each containing a single gene, at each step in the iteration the two closest clusters were merged into a larger cluster. The distance between clusters was defined as the distance between their average expression patterns. After N-1 steps all the data points were merged together. The clustering process defines a hierarchical tree. Genes were automatically assigned to a cluster by cutting the tree between the root and each gene branch with a set of 10 lines (“branch levels”) separated by fixed distances. The branch level cut-off forms a cluster. The tree was first ‘normalized’ so that each branch was at the same distance from the root. In order to preserve the distance between the closest genes, the tree was distorted at the branch furthest from the leaf. The number of branches intersecting at each branch level of the tree equals the number of clusters at that level.

Division of the tree at branch level 5 divides the genes into 7 clusters of gene expression which include 276 differentially expressed genes and splice variants. In tables 1, columns 4through 10 show the level of gene expression at each time point in response to Ox-LDL exposure vs. no Ox-LDL. Differential regulation has been normalized to a maximum value of 1.0 for each gene. White represents relative expression in response to Ox-LDL ranging from 0-25% of maximum for that particular gene; light gray from 26-50%; dark gray from 51-75%; black from 76-100%.

IX. Complementary Nucleic Acid Molecules

Molecules complementary to the polynucleotide, or a fragment thereof, are used to detect, decrease, or inhibit gene expression Although use of oligonucleotides comprising from about 15 to about 30 base pairs is described, the same procedure is used with larger or smaller fragments or their derivatives (PNAs). Oligonucleotides are selected using OLIGO 4.06 software (National Biosciences) and SEQ ED NOs:1-278. To inhibit transcription by preventing promoter binding, a complementary oligonucleotide is designed to bind to the most unique 5′ sequence, most preferably about 10 nucleotides before the initiation codon of the open reading frame. To inhibit translation, a complementary oligonucleotide is designed to prevent ribosomal binding to the mRNA encoding the protein.

In addition to using antisense molecules constructed to interrupt transcription or translation, modifications of gene expression can be obtained by designing antisense molecules to genomic sequences (such as enhancers or introns) or even to trans-acting regulatory genes. Similarly, antisense inhibition can be achieved using Hogeboom base-pairing methodology, also known as “triple helix” base pairing. Antisense molecules involved in triple helix pairing compromise the ability of the double helix to open sufficiently for the binding of polymerases, transcription factors, or regulatory molecules.

Such antisense molecules are placed in expression vectors and used to transform preferred cells or tissues. This may include introduction of the expression vector into a cell line to test efficacy; into an organ, tumor, synovial cavity, or the vascular system for transient or short term therapy; or into a stem cell or other reproducing lineage for long term or stable gene therapy. Transient expression may last for a month or more with a non-replicating vector and for three months or more if appropriate elements for inducing vector replication are used in the transformation/expression system.

Stable transformation of appropriate dividing cells with a vector encoding the antisense molecule can produce a transgenic cell line, tissue, or organism (U.S. Pat. No. 4,736,866). Those cells that assimilate and replicate sufficient quantities of the vector to allow stable integration also produce enough antisense molecules to compromise or entirely eliminate activity of the polynucleotide.

X. Hybridization Technologies and Analyses

Hybridization technology utilizes a variety of substrates such as polymer coated glass slides and nylon membranes. Arranging elements on polymer coated slides is described in Example V; probe preparation and hybridization and analysis using polymer coated slides is described in examples VI and VII, respectively.

Polynucleotides are applied to a membrane substrate by one of the following methods. A mixture of polynucleotides is fractionated by gel electrophoresis and transferred to a nylon membrane by capillary transfer. Alternatively, the polynucleotides are individually ligated to a vector and inserted into bacterial host cells to form a library. The polynucleotides are then arranged on a substrate by one of the following methods. In the first method, bacterial cells containing individual clones are robotically picked and arranged on a nylon membrane. The membrane is placed on LB agar containing selective agent (carbenicimin, kanamycin, ampicillin, or cnloramphenicol depending on the vector used) and incubated at 37° C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, polynucleotides are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL400 beads (Amersham Pharmacia Biotech). Purified nucleic acids are arranged on a nylon membrane manually or using a dot/slot blotting manifold and suction device and are immobilized by denaturation, neutralization, and UV irradiation as described above.

Hybridization probes derived from polynucleotides of the Sequence Listing are employed for screening cDNAs, mRNAs, or genomic DNA in membrane-based hybridizations. Probes are prepared by diluting the polynucleotides to a concentration of 40-50 ng in 45 μl TE buffer, denaturing by heating to 100° C. for five min, and briefly centrifuging. The denatured polynucleotide is then added to a REDIPRIME tube (Amersham Pharmacia Biotech), gently mixed until blue color is evenly distributed, and briefly centrifuged. Five microliters of [³²P]dCTP is added to the tube, and the contents are incubated at 37° C. for 10 min. The labeling reaction is stopped by adding 5 μl of 0.2M EDTA, and probe is purified from unincorporated nucleotides using a PROBEQUANT G-50 microcolumn (Amersham Pharmacia Biotech). The purified probe is heated to 100° C. for five min, snap cooled for two min on ice.

Membranes are pre-hybridized in hybridization solution containing 1% Sarkosyl and 1× high phosphate buffer (0.5 M NaCl, 0.1 M Na₂HPO₄, 5 mM EDTA, pH 7) at 55° C. for two hr. The probe, diluted in 15 ml fresh hybridization solution, is then added to the membrane. The membrane is hybridized with the probe at 55° C. for 16 hr. Following hybridization, the membrane is washed for 15 min at 25° C. in 1 mM Tris (pH 8.0), 1% Sarkosyl, and four times for 15 min each at 25° C. in 1 mM Tris (pH 8.0). To detect hybridization complexes, XOMAT-AR film (Eastman Kodak, Rochester N.Y.) is exposed to the membrane overnight at −70° C., developed, and examined visually.

XI. Expression of the Encoded Protein

Expression and purification of a protein encoded by a polynucleotide of the invention is achieved using bacterial or virus-based expression systems. For expression in bacteria, cDNA is subcloned into a vector containing an antibiotic resistance gene and an inducible promoter that directs high levels of cDNA transcription Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21(DE3). Antibiotic resistant bacteria express the protein upon induction with isopropyl beta-D-thiogalactopyranoside (IPTG). Expression in eukaryotic cells is achieved by infecting Spodoptera frugiperda (Sf9) insect cells with recombinant baculovirus, Autogaphica californica nuclear polyhedrosis virus. The polyhedrin gene of baculovirus is replaced with the polynucleotide by either homologous recombination or bacterial-mediated transposition involving transfer plasmid intermediates. Viral infectivity is maintained and the strong polyhedrin promoter drives high levels of polynucleotide transcription.

For ease of purification, the protein is synthesized as a fusion protein with glutathione-S-transferase (GST; Amersham Pharmacia Biotech) or a similar alternative such as FLAG. The fusion protein is purified on immobilized glutathione under conditions that maintain protein activity and antigenicity. After purification, the GST moiety is proteolytically cleaved from the protein with thrombin. A fusion protein with FLAG, an 8-amino acid peptide, is purified using commercially available monoclonal and polyclonal anti-FLAG antibodies (Eastman Kodak, Rochester N.Y.).

XII. Production of Specific Antibodies

A denatured polypeptide from a reverse phase HPLC separation is obtained in quantities up to 75 mg. This denatured protein is used to immunize mice or rabbits following standard protocols. About 100 μg is used to immunize a mouse, while up to 1 mg is used to immunize a rabbit. The denatured polypeptide is radioiodinated and incubated with murine B-cell hybridomas to screen for monoclonal antibodies. About 20 mg of polypeptide is sufficient for labeling and screening several thousand clones.

In another approach, the amino acid sequence translated from a polynucleotide of the invention is analyzed using PROTEAN software (DNASTAR) to determine regions of high inmunogenicity. The optimal sequences for immunization are usually at the C-terminus, the N-terminus, and those intervening, hydrophilic regions of the polypeptide that are likely to be exposed to the external environment when the polypeptide is in its natural conformation. Typically, oligopeptides about 15 residues in length are synthesized using an ABI 431 Peptide synthesizer (PE Biosystems) using Fmoc-chemistry and then coupled to keyhole limpet hemocyanin (KLH; Sigma Aldrich) by reaction with M-maleimidobenzoyl-N-hydroxysuccinimide ester. If necessary, a cysteine may be introduced at the N-terminus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated polypeptide to identify those fusions producing a monoclonal antibody specific for the polypeptide. In a typical protocol, wells of 96 well plates (FAST, Becton-Dickinson, Palo Alto Calif.) are coated with affinity-purified, specific rabbit-anti-mouse (or suitable anti-species Ig) antibodies at 10 mg/ml. The coated wells are blocked with 1% BSA and washed and exposed to supernatants from hybridomas. After incubation, the wells are exposed to radiolabeled polypeptide at 1 mg/ml. Clones producing antibodies bind a quantity of labeled polypeptide that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on protein A (Amersham Pharmacia Biotech). Monoclonal antibodies with affinities of at least 10⁸ M⁻¹, preferably 10⁹ to 10¹⁰ M⁻¹or stronger, are made by procedures well known in the art.

XIH. Purification of Naturally Occurring Protein Using Specific Antibodies

Naturally occurring or recombinant protein is substantially purified by immunoaffinity chromatography using antibodies specific for the protein. An immunoaffinity column is constructed by covalently coupling the antibody to CNBr-activated SEPHAROSE resin (Amersham Pharmacia Biotech). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV. Screening Molecules for Specific Binding

The polynucleotide or fragments thereof are labeled with ³²P-dCTP, Cy3-dCTP, Cy5-dCTP (Amersham Pharmacia Biotech), or the protein or portions thereof are labeled with BIODIPY or FITC (Molecular Probes). A library or a plurality of candidate molecules or compounds previously arranged on a substrate are incubated in the presence of labeled polynucleotide or protein. After incubation under conditions for a polynucleotide or protein, the substrate is washed. Any position on the substrate retaining label, that indicates specific binding or complex formation, identifies a ligand. Data obtained using different concentrations of the polynucleotide or polypeptide are used to calculate affinity between the labeled polynucleotide or protein and the bound ligand.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

TABLE 1

| SEQ ID NO | Incyte ID | Gene Annotation | 0 h | 0.5 h | 2.5 h | 8 h | 1 d | 2 d | 4 d | Cluster |
|---|---|---|---|---|---|---|---|---|---|---|
| 1 | 440295.1 | Human SBC2 mRNA for sodium bicarbonate transporter 2, complete cds. | 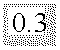 | 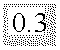 | 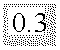 | 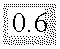 | 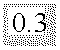 | 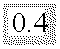 | 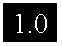 | 1 |
| 2 | g34387 | annexin 1 (lipocortin I) | 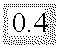 | 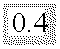 | 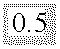 | 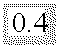 | 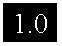 | 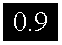 | 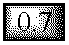 | 2 |
| 3 | 247178.2 | sperm surface protein | 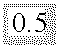 | 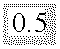 | 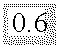 | 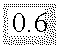 | 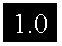 | 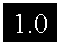 | 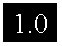 | 2 |
| 4 | 567938 | integrin, alpha X (antigen CD11C (p150), alpha polypeptide) | 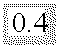 | 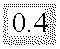 | 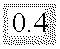 | 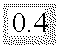 | 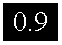 | 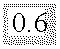 | 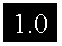 | 2 |
| 5 | 351122.2 | integrin, beta 3 (platelet glycoprotein IIIa, antigen CD61) | 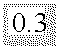 | 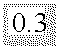 | 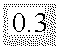 | 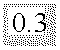 | 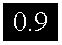 | 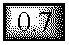 | 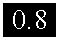 | 2 |
| 6 | 481379.9 | paired basic amino acid cleaving enzyme (furin, membrane associated receptor protein) | 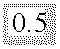 | 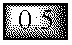 | 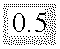 | 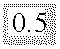 | 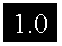 | 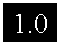 | 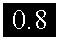 | 2 |
| 7 | 215391.7 | phosphogluconate dehydrogenase | 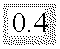 | 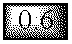 | 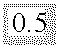 | 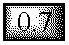 | 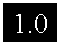 | 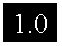 | 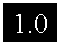 | 2 |
| 8 | 243812.1 | protein kinase mitogen-activated 13 | 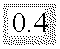 | 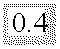 | 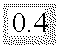 | 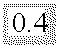 | 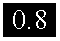 | 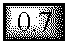 | 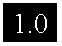 | 2 |
| 9 | 1085755.1 | folate receptor 1 (adult) | 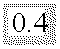 | 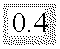 | 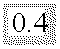 | 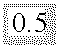 | 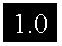 | 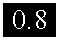 | 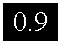 | 2 |
| 10 | 347809.3 | solute carrier family 6 (neurotransmitter transporter, taurine), member 6 | 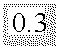 | 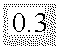 | 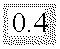 | 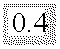 | 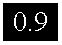 | 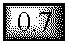 | 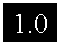 | 2 |
| 11 | 331734.4 | prostaglandin-endoperoxide synthase 1 (prostaglandin G/H synthase and cyclooxygenase) | 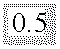 | 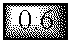 | 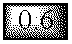 | 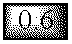 | 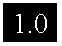 | 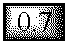 | 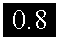 | 2 |
| 12 | 116840.38 | interferon regulatory factor 3 | 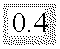 | 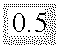 | 0.4 | 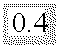 | 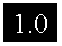 | 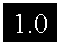 | 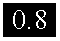 | 2 |
| 13 | 903565.11 | proprotein convertase subtilisin/kexin type 4 | 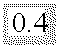 | 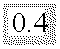 | 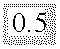 | 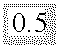 | 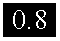 | 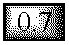 | 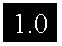 | 2 |
| 14 | 903565.8 | Human mRNA for PACE4E-I, complete cds. | 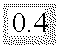 | 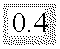 | 0.5 | 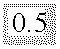 | 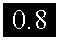 | 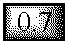 | 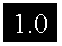 | |
| 15 | 474310.13 | transglutaminase 2 (C polypeptide, protein-glutamine-gamma-glutamyltransferase) | 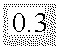 | 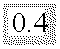 | 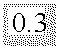 | 0.2 | 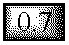 | 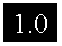 | 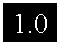 | 2 |
| 16 | 413006.13 | differentiated Embryo Chondrocyte expressed gene 1 | 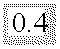 | 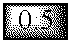 | 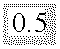 | 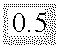 | 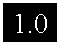 | 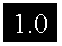 | 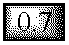 | 2 |
| 17 | 76460.2 | pyridoxal (pyridoxine, vitamin B6) kinase | 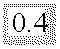 | 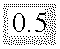 | 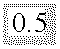 | 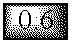 | 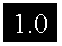 | 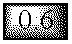 | 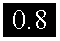 | 2 |
| 18 | 474374.4 | pim-1 oncogene | 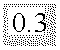 | 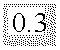 | 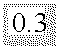 | 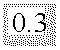 | 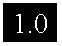 | 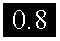 | 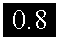 | 2 |
| 19 | 427792.8 | cathepsin B | 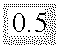 | 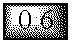 | 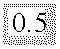 | 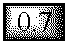 | 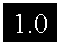 | 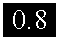 | 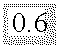 | 2 |
| 20 | 364482.3 | carnitine palmitoyltransferase I, liver | 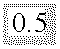 | 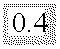 | 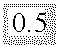 | 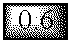 | 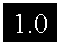 | 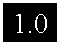 | 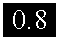 | 2 |
| 21 | 978487.1 | carnitine palmitoyltranserase I, liver | 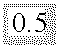 | 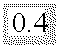 | 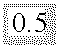 | 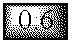 | 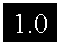 | 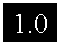 | 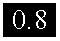 | 2 |
| 22 | 410626.2 | Human retinoid X receptor-gamma mRNA, complete cds | 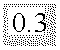 | 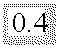 | 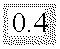 | 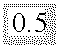 | 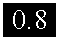 | 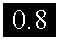 | 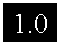 | 2 |
| 23 | 234480.6 | glutaredoxin (thioltransferase) | 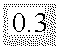 | 0.2 | 0.2 | 0.2 | 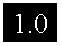 | 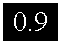 | 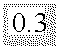 | 3 |
| 24 | 253542.2 | dual specificity phosphatase 5 | 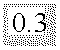 | 0.2 | 0.2 | 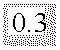 | 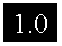 | 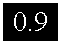 | 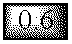 | 3 |
| 25 | 234202.24 | microsomal glutathione S-transferase 1 | 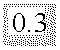 | 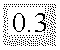 | 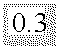 | 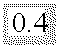 | 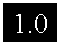 | 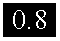 | 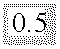 | 3 |
| 26 | 253946.4 | interleukin 6 signal transducer (gp130, oncostatin M receptor) | 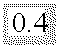 | 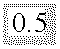 | 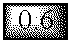 | 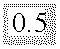 | 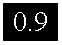 | 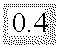 | 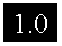 | 3 |
| 27 | 348801.1 | pro-platelet basic protein | 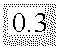 | 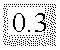 | 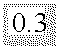 | 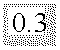 | 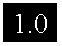 | 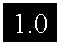 | 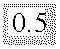 | 3 |
| 28 | 980611.1 | matrilin 1, cartilage matrix protein | 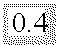 | 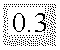 | 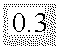 | 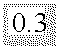 | 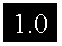 | 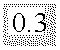 | 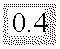 | 3 |
| 29 | 283885.8 | dual-specificity tyrosine-(Y)-phosphorylation regulated kinase 4 | 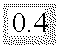 | 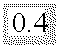 | 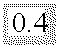 | 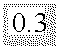 | 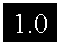 | 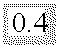 | 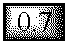 | |
| 30 | 348196.33 | antigen identified by monoclonal antibodies 4F2, TRA1.10, TROP4, and T43 | 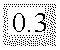 | 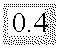 | 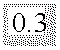 | 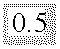 | 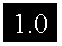 | 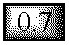 | 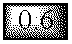 | 3 |
| 31 | 256009.4 | AHNAK nucleoprotein (desmoyokin) | 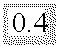 | 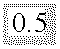 | 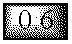 | 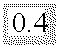 | 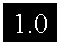 | 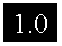 | 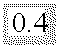 | 3 |
| 32 | 481594.12 | Human RACH1 (RACH1) mRNA, complete cds | 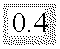 | 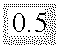 | 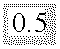 | 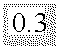 | 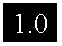 | 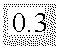 | 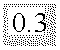 | 3 |
| 33 | 978788.1 | Human RACH1 (RACH1) mRNA, complete cds | 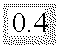 | 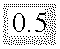 | 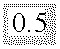 | 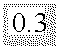 | 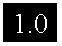 | 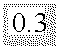 | 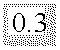 | 3 |
| 34 | 335171.1 | integrin, alpha 2 (CD49B, alpha 2 subunit of VLA-2 receptor) | 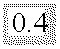 | 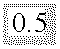 | 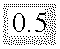 | 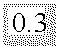 | 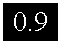 | 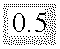 | 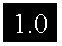 | 3 |
| 35 | 998433.2 | ESTs, Highly similar to DIAMINE ACETYLTRANSFERASE ([H. sapiens] | 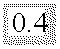 | 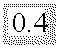 | 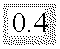 | 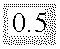 | 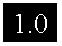 | 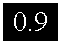 | 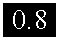 | 2 |
| 36 | 221928.9 | ESTs | 0.3 | 0.3 | 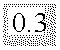 | 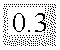 | 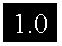 | 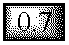 | 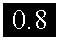 | 2 |
| 37 | 331291.3 | Homo sapiens mRNA for KIAA0291 gene, partial cds | 0.4 | 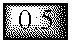 | 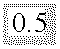 | 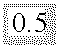 | 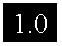 | 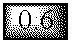 | 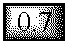 | 2 |
| 38 | 233331.3 | Homo sapiens KIAA0439 mRNA, partial cds | 0.4 | 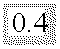 | 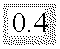 | 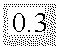 | 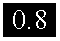 | 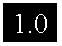 | 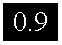 | 2 |
| 39 | 474682.2 | ESTs, Weakly similar to W01A11.2 gene product [C. elegans] | 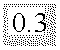 | 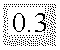 | 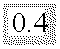 | 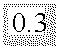 | 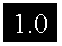 | 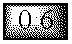 | 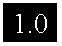 | 2 |
| 40 | 3161.7 | ESTs, Weakly similar to (define not available 4529890) [H. sapiens] | 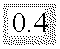 | 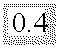 | 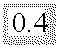 | 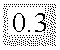 | 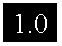 | 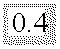 | 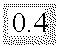 | 3 |
| 41 | 984248.1 | ESTs | 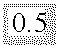 | 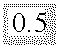 | 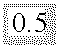 | 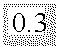 | 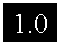 | 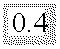 | 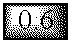 | 3 |
| 42 | 196590.2 | ESTs | 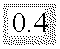 | 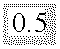 | 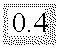 | 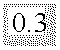 | 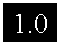 | 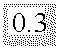 | 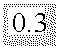 | 3 |
| 43 | 255109.1 | ESTs | 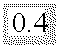 | 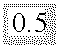 | 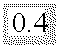 | 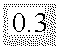 | 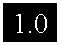 | 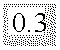 | 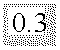 | 3 |
| 44 | 238622.2 | Human clone 46690 brain expressed mRNA from Chromosome X | 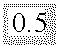 | 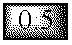 | 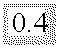 | 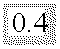 | 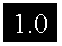 | 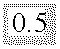 | 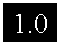 | 3 |
| 45 | 334385.3 | Homo sapiens mRNA for KIAA0284 gene, partial cds | 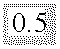 | 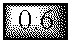 | 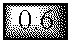 | 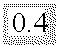 | 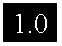 | 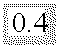 | 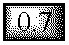 | 3 |
| 46 | 998997.1 | ESTs | 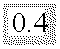 | 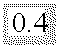 | 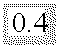 | 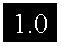 | 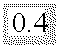 | 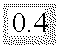 | 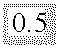 | 7 |
| 47 | 200578.1 | ESTs | 0.2 | 0.2 | 0.2 | 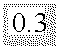 | 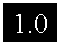 | 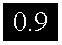 | 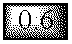 | 3 |
| 48 | 208134.1 | ESTs | 0.2 | 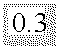 | 0.2 | 0.2 | 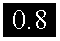 | 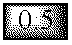 | 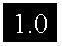 | 3 |
| 49 | 153659.2 | interleukin 1 receptor antagonist | 0.2 | 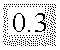 | 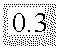 | 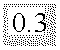 | 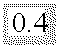 | 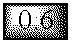 | 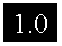 | 1 |
| 50 | 241930.15 | liver X receptor, alpha | 0.2 | 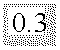 | 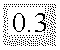 | 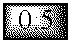 | 0.4 | 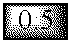 | 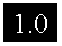 | 1 |
| 51 | 413466.5 | adipose differentiation-related protein; adipophilin | 0.1 | 0.1 | 0.2 | 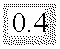 | 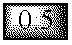 | 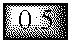 | 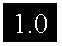 | 1 |
| 52 | 3249239 | colony stimulating factor 1 (macrophage) | 0.2 | 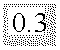 | 0.2 | 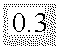 | 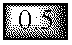 | 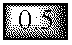 | 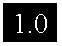 | 1 |
| 53 | 337518.18 | CD36 antigen (collagen type I receptor, thrombospondin receptor) | 0.2 | 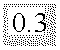 | 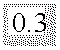 | 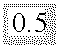 | 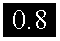 | 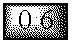 | 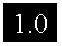 | 2 |
| 54 | g3116213 | SH3 binding protein | 0.2 | 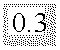 | 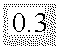 | 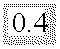 | 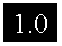 | 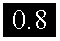 | 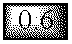 | 3 |
| 55 | g5912216 | SH3 binding protein | 0.2 | 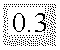 | 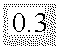 | 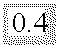 | 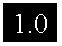 | 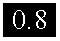 | 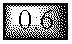 | 3 |
| 56 | 992917.1 | ferritin, heavy polypeptide 1 | 0.2 | 0.2 | 0.2 | 0.2 | 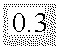 | 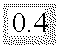 | 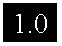 | 1 |
| 57 | 411424.12 | LIM and senescent cell antigen-like domains 1 | 0.2 | 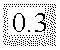 | 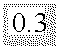 | 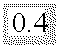 | 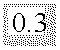 | 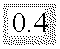 | 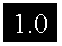 | 1 |
| 58 | 995600.17 | Homo sapiens clone 24649 mRNA sequence | 0.2 | 0.2 | 0.2 | 0.2 | 0.2 | 0.2 | 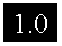 | 1 |
| 59 | 441292.7 | epithelial membrane protein 1 | 0.2 | 0.2 | 0.2 | 0.2 | 0.3 | 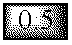 | 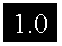 | 1 |
| 60 | 42176.5 | Down syndrome dandidate region 1 | 0.1 | 0.2 | 0.2 | 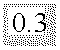 | 0.2 | 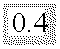 | 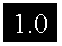 | 1 |
| 61 | 234537.3 | 5′ nucleotidase (CD73) | 0.2 | 0.2 | 0.2 | 0.2 | 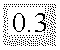 | 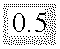 | 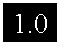 | 1 |
| 62 | 4704568.21 | uridine phosphorylase | 0.2 | 0.2 | 0.2 | 0.2 | 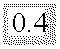 | 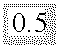 | 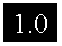 | 1 |
| 63 | 240120.3 | diphtheria toxin receptor (heparin-binding epidermal growth factor-like growth factor) | 0.1 | 0.1 | 0.1 | 0.1 | 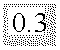 | 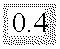 | 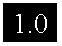 | 1 |
| 64 | 28779.3 | small inducible cytokine subfamily A (Cys-Cys), member 20 | 0.1 | 0.1 | 0.1 | 0.2 | 0.1 | 0.2 | 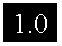 | 1 |
| 65 | 238627.2 | BCL2-related protein A1 | 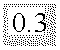 | 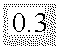 | 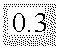 | 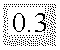 | 0.1 | 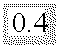 | 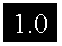 | 1 |
| 66 | 254107.1 | thrombomodulin | 0.2 | 0.2 | 0.1 | 0.1 | 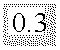 | 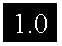 | 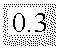 | 4 |
| 67 | 330908.2 | leukemia inhibitory factor (cholinergic differentiation factor) | 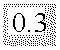 | 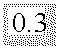 | 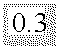 | 0.2 | 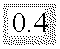 | 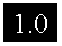 | 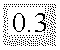 | 4 |
| 68 | g687589 | Human (AFlq) mRNA, complete cds | 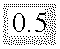 | 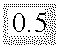 | 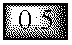 | 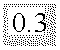 | 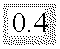 | 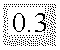 | 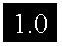 | 1 |
| 69 | 197975.11 | KIAA0763 gene product | 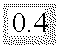 | 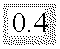 | 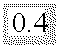 | 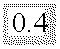 | 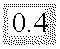 | 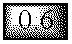 | 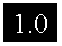 | 1 |
| 70 | 227928.2 | KIAA0429 gene product | 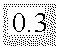 | 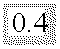 | 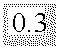 | 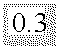 | 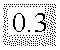 | 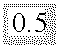 | 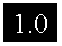 | 1 |
| 71 | 248785.7 | ESTs | 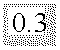 | 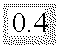 | 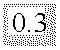 | 0.2 | 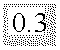 | 0.2 | 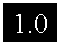 | 1 |
| 72 | 977757.3 | KIAA0237 gene product | 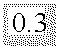 | 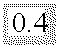 | 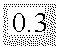 | 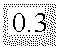 | 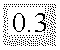 | 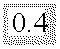 | 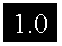 | 1 |
| 73 | 232773.2 | ESTs | 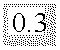 | 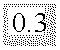 | 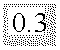 | 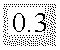 | 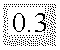 | 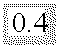 | 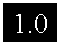 | 1 |
| 74 | g6634024 | Human mRNA for KIAA0379 gene, partial cds | 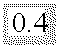 | 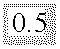 | 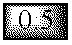 | 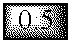 | 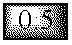 | 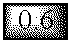 | 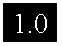 | 1 |
| 75 | g4589571 | ESTs, Weakly similar to DAP-1 beta [H. sapiens] | 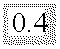 | 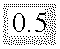 | 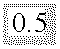 | 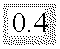 | 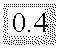 | 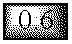 | 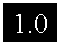 | 1 |
| 76 | 334370.3 | KIAA0024 gene product | 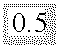 | 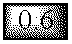 | 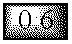 | 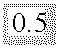 | 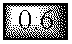 | 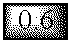 | 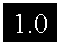 | 1 |
| 77 | 980461.1 | ESTs | 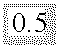 | 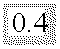 | 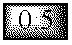 | 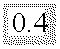 | 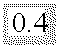 | 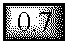 | 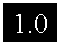 | 1 |
| 78 | 422969.5 | KIAA0598 gene product | 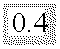 | 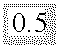 | 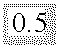 | 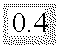 | 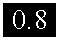 | 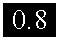 | 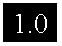 | 2 |
| 79 | 244150.4 | Human mRNA for KIAA0194 gene, partial cds | 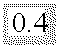 | 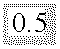 | 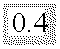 | 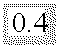 | 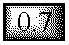 | 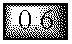 | 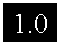 | 2 |
| 80 | 410257.11 | ESTs | 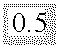 | 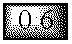 | 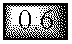 | 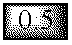 | 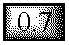 | 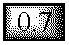 | 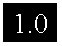 | 2 |
| 81 | 28253.3 | Homo sapiens chromosome 19, cosmid R28379 | 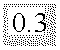 | 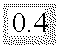 | 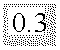 | 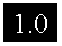 | 0.2 | 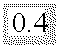 | 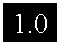 | 1 |
| 82 | g31670 | guanylate cyclase 1, soluble, alpha 2 | 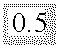 | 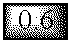 | 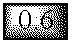 | 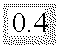 | 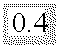 | 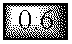 | 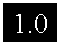 | 1 |
| 83 | 977552.1 | musculin (activated B-cell factor-1) | 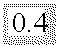 | 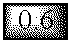 | 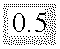 | 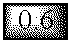 | 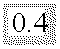 | 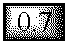 | 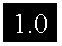 | 1 |
| 84 | 977552.2 | Human activated B-cell factor-1 (ABF-1) mRNA, complete cds. | 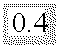 | 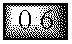 | 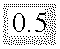 | 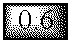 | 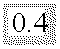 | 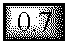 | 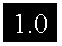 | 1 |
| 85 | 347829.6 | yes-associated protein 65 kDa | 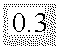 | 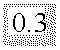 | 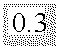 | 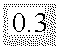 | 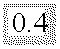 | 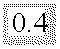 | 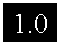 | 1 |
| 86 | 251776.11 | integrin, beta 5 | 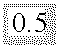 | 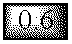 | 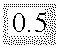 | 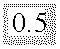 | 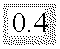 | 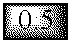 | 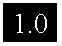 | 1 |
| 87 | 343674.9 | GTP-binding protein overexpressed in skeletal muscle | 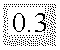 | 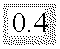 | 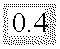 | 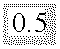 | 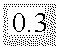 | 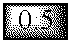 | 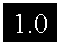 | 1 |
| 88 | 479136.1 | core-binding factor, runt domain, alpha subunit 3 | 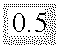 | 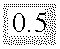 | 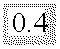 | 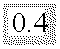 | 0.4 | 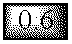 | 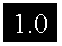 | 1 |
| 89 | 1078147.1 | early development regulator 2 (homolog of polyhomeotic 2) | 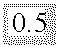 | 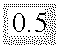 | 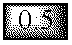 | 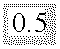 | 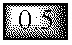 | 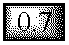 | 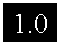 | 1 |
| 90 | 474275.1 | podocalyxin-like | 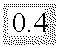 | 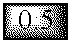 | 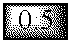 | 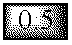 | 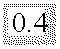 | 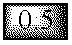 | 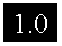 | 1 |
| 91 | 1320658 | fibulin 1 | 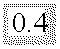 | 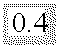 | 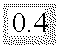 | 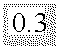 | 0.4 | 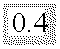 | 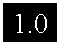 | 1 |
| 92 | 242114.16 | PTK2 protein tyrosine kinase 2 | 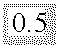 | 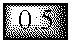 | 0.5 | 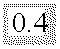 | 0.4 | 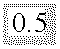 | 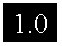 | 1 |
| 93 | 445186.7 | LIM domain only 4 | 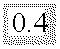 | 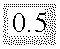 | 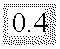 | 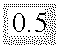 | 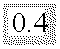 | 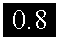 | 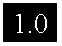 | 1 |
| 94 | 474496.2 | toll-like receptor 2 | 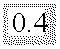 | 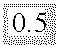 | 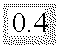 | 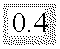 | 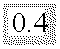 | 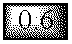 | 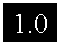 | 1 |
| 95 | 257114.7 | solute carrier family 31 (copper transporters), member 2 | 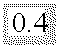 | 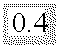 | 0.5 | 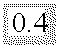 | 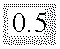 | 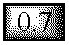 | 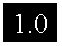 | 1 |
| 96 | 984005.1 | high-mobility group (nonhistone chromosomal) protein isoform I-C | 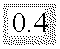 | 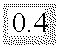 | 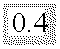 | 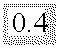 | 0.3 | 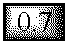 | 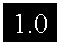 | 1 |
| 97 | 977667.1 | complement component 5 receptor 1 (C5a ligand) | 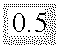 | 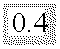 | 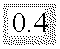 | 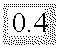 | 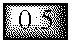 | 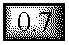 | 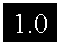 | 1 |
| 98 | 996862.4 | TG-interacting factor (TALE family homeobox) | 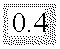 | 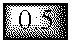 | 0.5 | 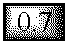 | 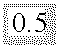 | 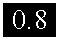 | 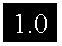 | 1 |
| 99 | 364940.19 | sparc/osteonectin, cwcv and kazal-like domains proteoglycan (testican) | 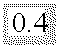 | 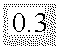 | 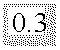 | 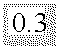 | 0.1 | 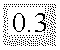 | 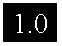 | 1 |
| 100 | 1041140.4 | Fc fragment of IgG, low affinity IIIa, receptor for (CD16) | 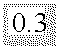 | 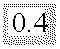 | 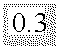 | 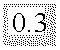 | 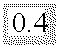 | 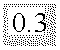 | 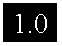 | 1 |
| 101 | 408246.2 | leupaxin | 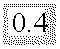 | 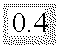 | 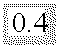 | 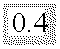 | 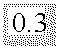 | 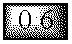 | 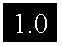 | 1 |
| 102 | 902740.4 | aminolevulinate, delta-, dehydratase | 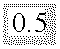 | 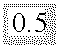 | 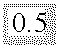 | 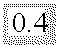 | 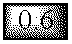 | 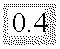 | 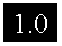 | 1 |
| 103 | 475486.9 | peptidylprolyl isomerase F (cyclophilin F) | 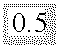 | 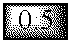 | 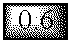 | 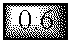 | 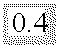 | 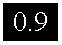 | 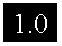 | 1 |
| 104 | 233778.9 | acid sphingomyelinase-like phosphodiesterase | 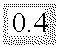 | 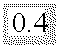 | 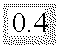 | 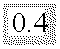 | 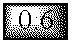 | 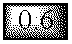 | 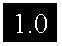 | 1 |
| 105 | 350392.3 | myosin IC | 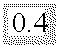 | 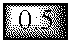 | 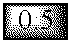 | 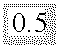 | 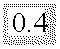 | 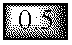 | 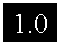 | 1 |
| 106 | 458045.4 | integrin, alpha 5 (fibronectin receptor, alpha polypeptide) | 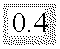 | 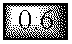 | 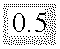 | 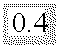 | 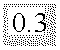 | 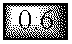 | 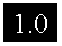 | 1 |
| 107 | 471362.17 | Homo sapiens myosin light chain kinase (MLCK) mRNA, complete cds | 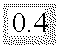 | 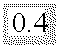 | 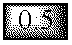 | 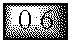 | 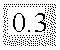 | 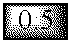 | 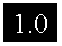 | 1 |
| 108 | 336716.3 | cytochrome P450, subfamily XXVIIB (25-hydroxyvitamin D-1-alpha-hydroxylase), polypeptide 1 | 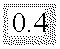 | 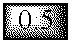 | 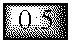 | 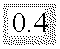 | 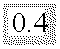 | 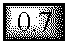 | 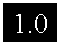 | 1 |
| 109 | 995211.5 | syndecan 2 (heparan sulfate proteoglycan 1, cell surface-associated, fibroglycan) | 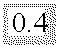 | 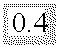 | 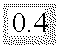 | 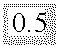 | 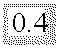 | 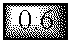 | 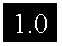 | 1 |
| 110 | 238824.2 | 3-prime-phosphoadenosine 5-prime-phosphosulfate synthase 1 | 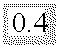 | 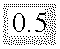 | 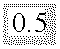 | 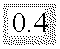 | 0.2 | 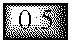 | 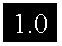 | 1 |
| 111 | 474592.3 | Human leukemia virus receptor 1 (GLVR1) mRNA, complete cds | 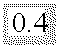 | 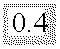 | 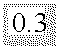 | 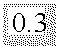 | 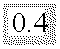 | 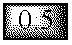 | 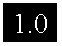 | 1 |
| 112 | 431338.2 | regulator of G-protein signaling 16 | 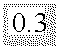 | 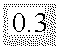 | 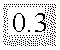 | 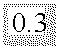 | 0.2 | 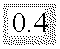 | 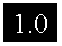 | 1 |
| 113 | 412631.5 | plectin 1, intermediate filament binding protein, 500 kD | 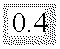 | 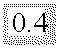 | 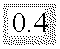 | 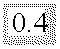 | 0.5 | 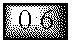 | 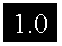 | 1 |
| 114 | 350480.6 | Gardner-Rasheed feline sarcoma viral (v-fgr) oncogene homolog | 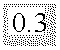 | 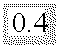 | 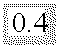 | 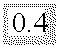 | 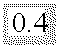 | 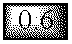 | 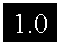 | 1 |
| 115 | 350521.15 | tumor necrosis factor receptor superfamily, member 10b | 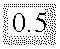 | 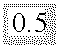 | 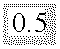 | 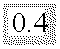 | 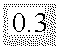 | 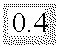 | 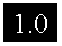 | 1 |
| 116 | 445076.9 | plasminogen activator, urokinase receptor | 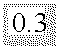 | 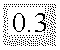 | 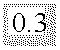 | 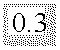 | 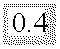 | 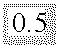 | 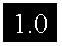 | 1 |
| 117 | 995028.4 | fibroblast activation protein, alpha | 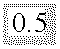 | 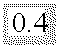 | 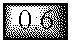 | 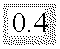 | 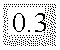 | 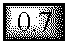 | 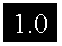 | 1 |
| 118 | 245008.4 | phosphodiesterase 8A | 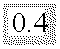 | 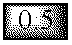 | 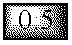 | 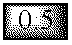 | 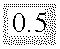 | 0.5 | 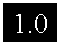 | 1 |
| 119 | 350895.1 | twist (Drosophila) homolog | 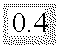 | 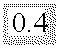 | 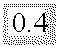 | 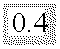 | 0.2 | 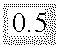 | 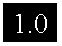 | 1 |
| 120 | 434265.5 | ribosomal protein S6 kinase, 90 kD, polypeptide 2 | 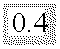 | 0.5 | 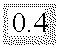 | 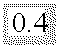 | 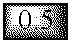 | 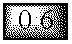 | 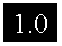 | 1 |
| 121 | 427813.14 | fibronectin 1 | 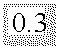 | 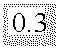 | 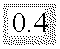 | 0.2 | 0.2 | 0.2 | 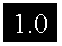 | 1 |
| 122 | 14704.3 | activin A receptor, type II | 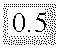 | 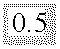 | 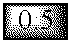 | 0.5 | 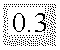 | 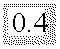 | 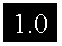 | 1 |
| 123 | 344240.2 | macrophage scavenger receptor 1 | 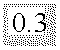 | 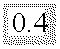 | 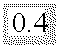 | 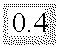 | 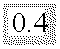 | 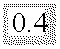 | 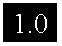 | 1 |
| 124 | 239694.6 | a disintegrin and metalloproteinase domain 17 (tumor necrosis factor, alph, converting enzyme) | 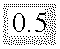 | 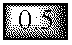 | 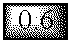 | 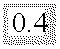 | 0.3 | 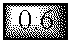 | 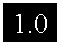 | 1 |
| 125 | 255772.2 | activin A receptor, type I | 0.5 | 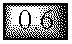 | 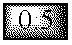 | 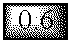 | 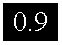 | 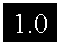 | 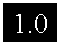 | 2 |
| 126 | 232066.3 | integrin, beta 7 | 0.5 | 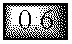 | 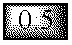 | 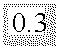 | 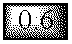 | 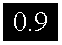 | 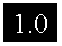 | 2 |
| 127 | 246504.1 | activating transcription factor 1 | 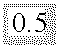 | 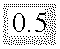 | 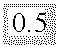 | 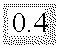 | 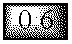 | 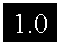 | 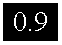 | 2 |
| 128 | 986123.22 | vimentin | 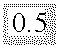 | 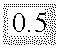 | 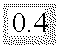 | 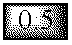 | 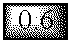 | 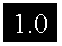 | 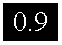 | 2 |
| 129 | 898945.14 | kynurenine 3-monooxygenase (kynurenine 3-hydroxylase) | 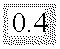 | 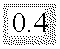 | 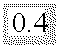 | 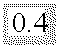 | 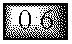 | 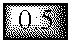 | 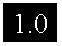 | 2 |
| 130 | 236208.16 | peptidylglycine alpha-amidating monooxygenase | 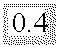 | 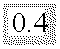 | 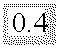 | 0.4 | 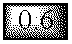 | 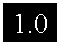 | 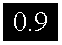 | 2 |
| 131 | 246531.2 | hippocalcin-like 1 | 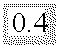 | 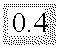 | 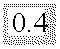 | 0.4 | 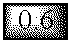 | 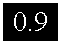 | 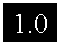 | 2 |
| 132 | 238586.2 | matrix metalloproteinase 7 (matrilysin, uterin) | 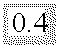 | 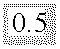 | 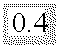 | 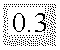 | 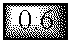 | 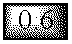 | 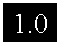 | 2 |
| 133 | 245532.7 | cyclin-dependent kinase inhibitor 1A (p21, Cip1) | 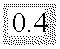 | 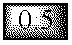 | 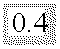 | 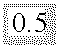 | 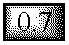 | 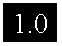 | 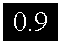 | 2 |
| 134 | 200972.2 | Human putative cyclin G1 interacting protein mRNA, partial sequence | 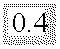 | 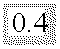 | 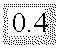 | 0.3 | 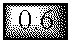 | 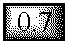 | 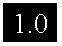 | 2 |
| 135 | 348061.1 | glucan (1,4-alpha-), branching enzyme 1 | 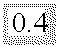 | 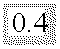 | 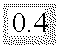 | 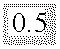 | 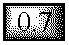 | 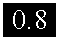 | 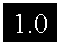 | 2 |
| 136 | 233711.7 | pyruvate dehydrogenase kinase, isoenzyme 4 | 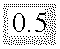 | 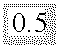 | 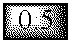 | 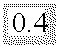 | 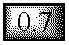 | 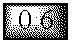 | 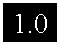 | 2 |
| 137 | 256043.19 | cathepsin L | 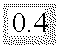 | 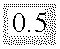 | 0.4 | 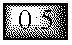 | 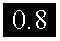 | 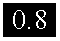 | 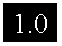 | 2 |
| 138 | 445012.6 | N-deacetylase/N-sulfotransferase (heparan glucosaminyl) 1 | 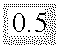 | 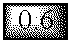 | 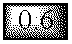 | 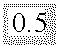 | 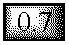 | 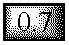 | 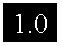 | 2 |
| 139 | g463906 | syntaxin 4A (placental) | 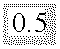 | 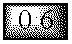 | 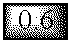 | 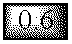 | 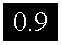 | 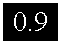 | 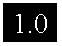 | 2 |
| 140 | 475621.1 | CD36 antigen (collagen type I receptor, thrombospondin receptor)-like 2 | 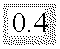 | 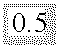 | 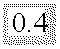 | 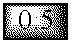 | 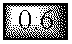 | 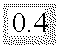 | 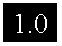 | 2 |
| 141 | 216063.17 | Human lysophospholipase homolog (HU-K5) mRNA, complete cds | 0.4 | 0.4 | 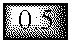 | 0.4 | 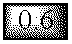 | 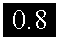 | 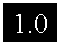 | 2 |
| 142 | 1099498.9 | apoliproprotein C-I | 0.5 | 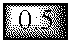 | 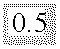 | 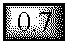 | 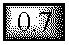 | 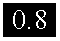 | 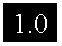 | 2 |
| 143 | 1099076.1 | fatty acid binding protein 5 (psoriasis-associated) | 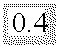 | 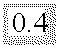 | 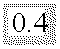 | 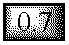 | 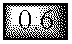 | 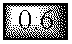 | 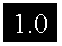 | 2 |
| 144 | 902119.3 | CD63 antigen (melanoma 1 antigen) | 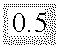 | 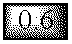 | 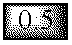 | 0.5 | 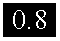 | 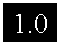 | 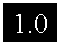 | 2 |
| 145 | g2982500 | neutropathy target esterase | 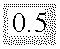 | 0.5 | 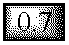 | 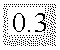 | 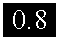 | 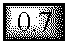 | 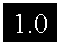 | 2 |
| 146 | 1097580.4 | ras homolog gene family, member C | 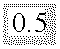 | 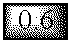 | 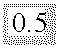 | 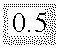 | 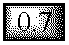 | 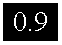 | 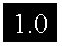 | 2 |
| 147 | 391851.1 | ferritin, light polypeptide | 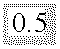 | 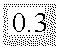 | 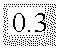 | 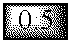 | 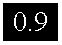 | 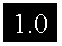 | 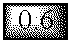 | 2 |
| 148 | 13105.9 | lectin, galactoside-binding, soluble, 3 (galectin 3) | 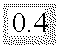 | 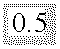 | 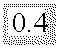 | 0.5 | 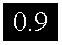 | 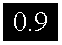 | 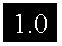 | 2 |
| 149 | 356248.4 | inositol phosphate 5′-phosphatase 2 (synaptojanin 2) | 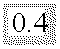 | 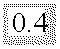 | 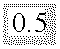 | 0.3 | 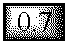 | 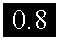 | 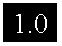 | 2 |
| 150 | 331045.1 | phosphodiesterase 3B, cGMP-inhibited | 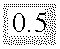 | 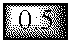 | 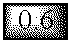 | 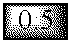 | 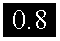 | 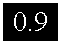 | 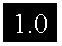 | 2 |
| 151 | 42480.3 | guanine nucleotide-releasing factor 2 (specific for crk proto-oncogene) | 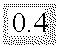 | 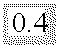 | 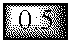 | 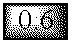 | 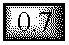 | 0.5 | 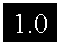 | 2 |
| 152 | 245099.8 | target of myb1 (chicken) homolog | 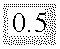 | 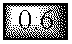 | 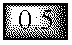 | 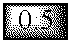 | 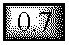 | 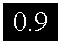 | 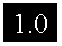 | 2 |
| 153 | 245481.2 | ciliary neurotrophic factor receptor | 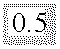 | 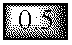 | 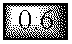 | 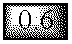 | 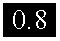 | 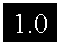 | 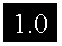 | 2 |
| 154 | 225021.4 | Burkitt lymphoma receptor 1, GTP-binding protein | 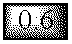 | 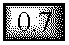 | 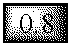 | 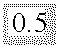 | 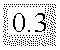 | 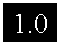 | 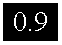 | 4 |
| 155 | 451767.28 | tissue inhibitor of metalloproteinase 3 (Sorsby fundus dystrophy, pseudoinflammatory) | 0.5 | 0.5 | 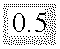 | 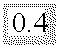 | 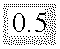 | 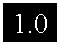 | 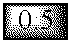 | 4 |
| 156 | 902142.11 | Homo sapiens leucocyte immunoglubulin-like receptor-5 (LIR-5) mRNA, complete cds | 0.5 | 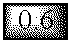 | 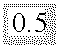 | 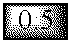 | 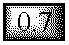 | 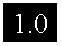 | 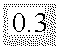 | 4 |
| 157 | 291095.5 | cytochrome P450, subfamily I (dioxin-inducible), polypeptide 1 (glaucoma 3, primary infantile) | 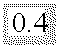 | 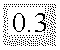 | 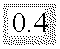 | 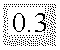 | 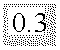 | 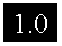 | 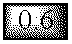 | 4 |
| 158 | 332919.4 | H. sapiens mRNA for cytokine inducible nuclear protein | 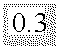 | 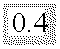 | 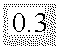 | 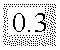 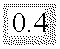 | 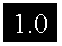 | 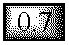 | 4 | |
| 159 | 387130.26 | choline kinase-like | 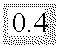 | 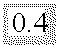 | 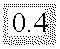 | 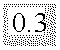 | 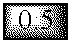 | 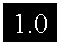 | 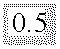 | 4 |
| 160 | 410580.13 | plasminogen activator inhibitor, type I | 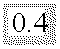 | 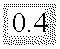 | 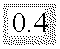 | 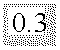 | 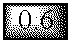 | 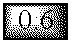 | 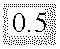 | 4 |
| 161 | 251715.1 | early growth response 1 | 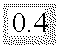 | 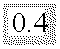 | 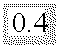 | 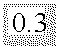 | 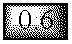 | 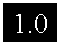 | 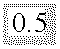 | 4 |
| 162 | 1799017F6 | neuregulin 1 | 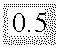 | 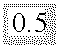 | 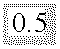 | 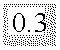 | 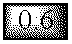 | 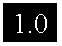 | 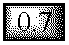 | 4 |
| 163 | 348891.1 | BCL2/adenovirus E1B 19 kD-interacting protein 3-like | 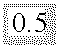 | 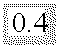 | 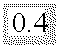 | 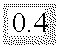 | 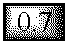 | 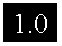 | 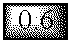 | 4 |
| 164 | 903956.15 | numb (Drosophila) homolog | 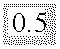 | 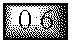 | 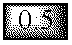 | 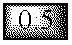 | 0.5 | 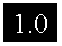 | 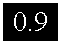 | 4 |
| 165 | 235184.1 | guanine nucleotide binding protein 11 | 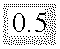 | 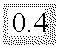 | 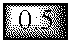 | 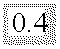 | 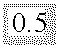 | 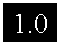 | 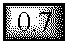 | 4 |
| 166 | 330948.3 | solute carrier family 9 (sodium/hydrogen exchanger), isoform 1 | 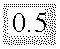 | 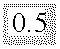 | 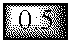 | 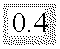 | 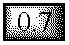 | 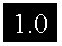 | 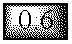 | 4 |
| 167 | 994057.1 | thrombospondin 1 | 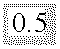 | 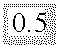 | 0.5 | 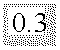 | 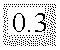 | 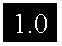 | 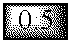 | 4 |
| 168 | 197301.4 | phosphoprotein regulated by mitogenic pathways | 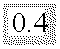 | 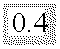 | 0.3 | 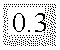 | 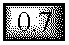 | 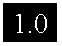 | 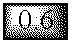 | 4 |
| 169 | 476016.17 | nuclear factor of kappa light polypeptide gene enhancer in B-cells inhibitor, alphs | 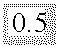 | 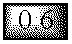 | 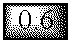 | 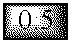 | 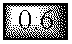 | 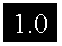 | 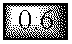 | 4 |
| 170 | 1098409.1 | early growth response 2 (Krox-20 (Drosophila) homolog) | 0.4 | 0.5 | 0.4 | 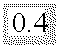 | 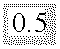 | 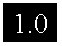 | 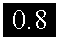 | 4 |
| 197 | 997377.1 | ribonuclease, RNase A family, 3 (eosinophil cationic protein) | 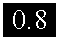 | 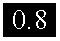 | 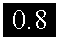 | 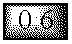 | 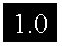 | 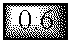 | 0.2 | 3 |
| 198 | 42869.3 | cathepsin G | 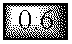 | 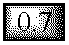 | 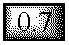 | 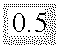 | 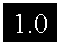 | 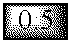 | 0.1 | 3 |
| 199 | 248306.1 | carbonic anhydrose II | 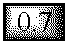 | 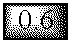 | 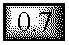 | 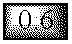 | 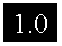 | 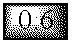 | 0.1 | 3 |
| 200 | 247220.15 | thymidylate synthetase | 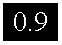 | 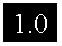 | 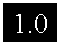 | 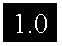 | 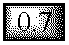 | 0.2 | 0.2 | 5 |
| 201 | 26662.3 | centromere protein F (350/400 kD, motosin) | 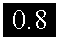 | 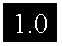 | 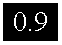 | 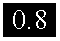 | 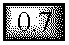 | 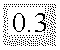 | 0.2 | 5 |
| 202 | 977509.3 | v-myb avian myeloblastosis viral oncogene homolog-like 2 | 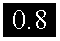 | 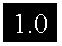 | 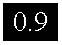 | 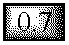 | 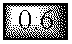 | 0.2 | 0.2 | 5 |
| 203 | 221961.2 | myeloid cell nuclear differentiation antigen | 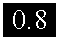 | 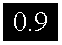 | 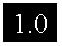 | 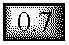 | 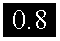 | 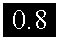 | 0.1 | 5 |
| 204 | 246824.1 | ribonuclease, RNase A family, 2 (liver, eosinophil-derived neurotoxin) | 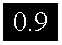 | 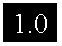 | 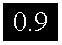 | 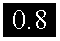 | 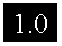 | 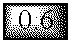 | 0.2 | 5 |
| 205 | 407557.2 | cyclin-dependent kinase inhibitor 2C (p18, inhibits CDK4) | 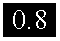 | 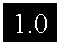 | 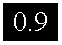 | 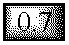 | 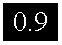 | 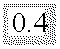 | 0.2 | 5 |
| 206 | 372981.2 | Homo sapiens ZW10 interactor Zwint mRNA, complete cds | 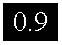 | 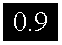 | 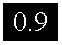 | 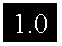 | 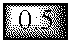 | 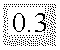 | 0.2 | 5 |
| 207 | 201409.6 | Fc fragment of IgG, high affinity Ia, receptor for (CD64) | 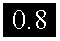 | 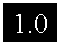 | 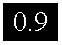 | 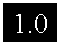 | 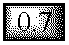 | 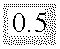 | 0.2 | 5 |
| 208 | 331025.1 | Homo sapiens mitotic centromere-associated kinesin mRNA, complete cds | 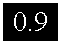 | 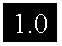 | 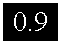 | 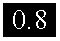 | 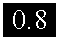 | 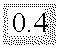 | 0.2 | 5 |
| 209 | 247515.1 | elastase 2, neutrophil | 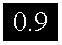 | 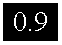 | 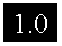 | 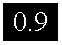 | 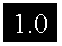 | 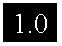 | 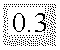 | 5 |
| 210 | 199471.2 | MAD2 (mitotic arrest deficient, yeast, homolog)-like 1 | 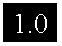 | 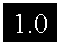 | 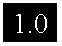 | 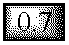 | 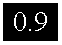 | 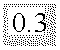 | 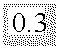 | 5 |
| 211 | 2916753 | high-mobility group (nonhistone chromosomal) protein 2 | 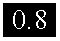 | 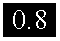 | 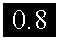 | 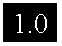 | 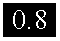 | 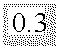 | 0.2 | 5 |
| 212 | 343899.2 | hyaluronan-mediated motility receptor (RHAMM) | 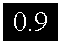 | 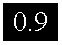 | 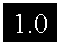 | 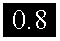 | 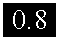 | 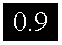 | 0.2 | 5 |
| 213 | 335775.2 | lamin B1 | 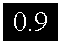 | 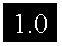 | 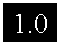 | 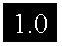 | 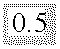 | 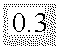 | 0.2 | 5 |
| 214 | 232714.5 | ESTs | 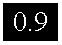 | 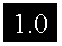 | 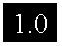 | 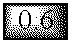 | 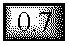 | 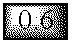 | 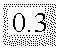 | 5 |
| 215 | 305039.4 | ESTs | 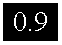 | 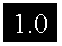 | 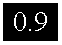 | 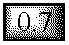 | 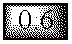 | 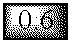 | 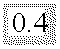 | 5 |
| 216 | 233603.2 | ESTs | 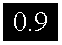 | 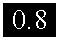 | 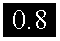 | 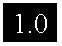 | 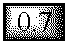 | 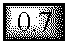 | 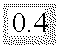 | 5 |
| 217 | 330930.1 | ESTs | 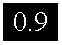 | 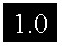 | 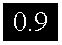 | 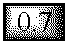 | 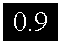 | 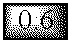 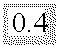 | 5 | |
| 218 | 247289.1 | Human clone 23815 mRNA sequence | 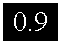 | 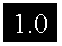 | 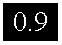 | 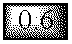 | 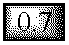 | 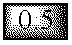 | 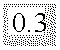 | 5 |
| 219 | 331033.1 | KIAA0008 gene product | 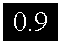 | 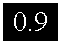 | 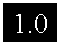 | 0.5 | 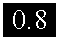 | 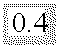 | 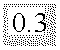 | 5 |
| 220 | 1098766.1 | ESTs | 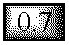 | 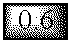 | 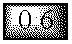 | 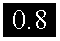 | 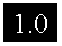 | 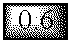 | 0.3 | 5 |
| 221 | 245632.3 | ESTs | 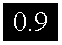 | 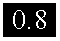 | 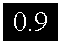 | 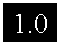 | 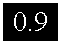 | 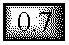 | 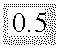 | 5 |
| 222 | 333461.2 | Human mRNA for KIAA0074 gene, partial cd | 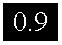 | 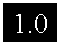 | 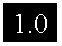 | 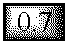 | 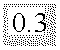 | 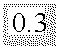 | 5 | |
| 223 | 347876.6 | minichromosome maintenance deficient (S. cerevisiae) 4 | 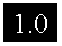 | 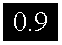 | 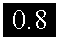 | 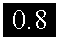 | 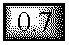 | 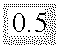 | 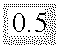 | 5 |
| 224 | 413842.1 | Human ECRP gene for eosinophil cationic related protein | 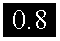 | 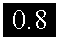 | 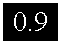 | 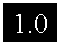 | 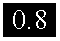 | 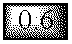 | 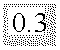 | 5 |
| 225 | 235867.2 | polo (Drosophia)-like kinase | 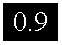 | 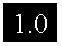 | 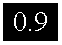 | 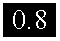 | 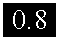 | 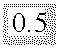 | 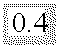 | 5 |
| 226 | 428665 | ribonucleotide reductase M1 polypeptide | 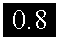 | 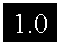 | 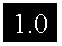 | 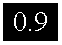 | 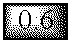 | 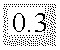 | 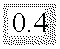 | 5 |
| 227 | 2234.3 | Homo sapiens histone H2A.F/Z variant (H2AV) mRNA, complete cds | 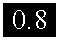 | 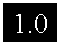 | 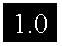 | 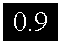 | 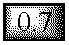 | 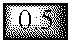 | 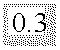 | 5 |
| 228 | 1000139.13 | insulin-like growth factor binding protein 7 | 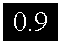 | 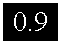 | 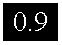 | 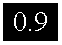 | 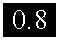 | 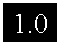 | 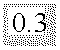 | 5 |
| 229 | 998534.1 | growth factor independent 1 | 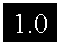 | 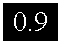 | 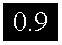 | 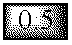 | 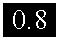 | 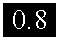 | 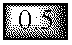 | 5 |
| 230 | 372377.6 | phosphorylase, glycogen; liver (Hers disease, glycogen storage disease type VI) | 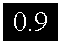 | 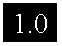 | 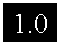 | 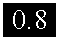 | 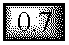 | 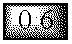 | 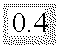 | 5 |
| 231 | 1101412.4 | trophinin-assisting protein (tastin) | 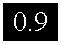 | 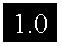 | 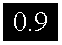 | 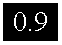 | 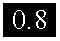 | 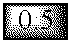 | 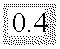 | 5 |
| 232 | 261567.5 | CDC28 protein kinase 2 | 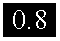 | 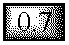 | 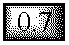 | 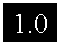 | 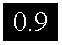 | 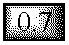 | 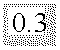 | 5 |
| 233 | 232713.2 | uracil-DNA glycosylase | 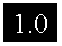 | 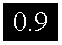 | 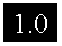 | 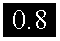 | 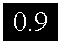 | 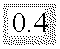 | 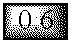 | 5 |
| 234 | 214335.13 | Homo sapiens E2F-related transcription factor (DP-1) mRNA, complete cds | 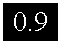 | 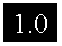 | 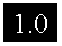 | 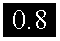 | 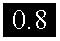 | 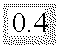 | 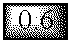 | 5 |
| 235 | 331022.33 | dihydropyrimidine dehydrogenase | 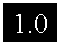 | 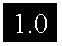 | 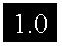 | 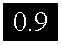 | 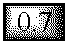 | 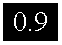 | 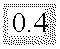 | 5 |
| 236 | 332259.3 | retinoblastoma-like 1 (p107) | 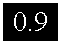 | 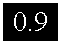 | 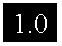 | 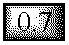 | 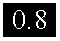 | 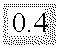 | 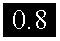 | 5 |
| 237 | 253570.8 | forkhead (Drosophila)-like 16 | 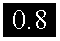 | 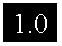 | 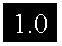 | 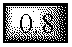 | 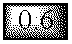 | 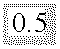 | 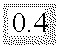 | 5 |
| 238 | 995529.5 | cell division cycle 2, G1 to S and G2 to M | 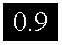 | 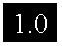 | 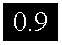 | 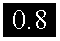 | 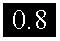 | 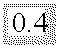 | 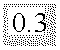 | 5 |
| 239 | 474435.16 | Human MAC30 mRNA, 3′ end | 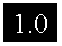 | 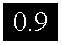 | 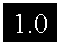 | 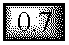 | 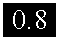 | 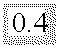 | 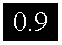 | 5 |
| 240 | 994861.1 | Human chondroitin sulfate proteoglycan core protein mRNA, 3′ end | 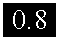 | 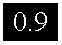 | 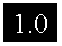 | 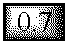 | 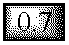 | 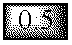 | 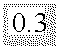 | 5 |
| 241 | g545708 | natural kill cell group 7 sequence | 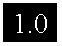 | 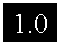 | 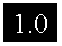 | 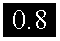 | 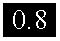 | 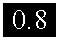 | 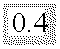 | 5 |
| 242 | 347965.2 | CD39 antigen | 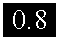 | 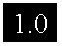 | 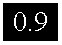 | 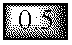 | 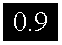 | 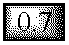 | 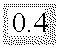 | 5 |
| 243 | 202361.1 | small nuclear ribonucleoprotein polypeptide A | 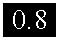 | 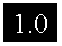 | 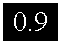 | 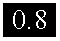 | 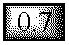 | 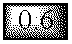 | 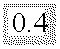 | 5 |
| 244 | 369950.12 | DNA-damage-inducible transcript 1 | 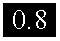 | 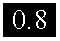 | 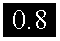 | 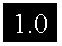 | 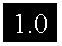 | 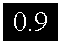 | 0.2 | 5 |
| 245 | 331403.8 | minichromosome maintenance deficient (S. cerevisiae) 5 (cell division cycle 46) | 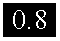 | 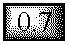 | 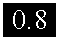 | 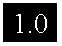 | 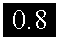 | 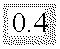 | 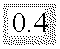 | 5 |
| 246 | 233889.3 | CDC28 proeint kinase 1 | 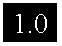 | 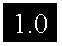 | 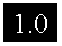 | 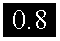 | 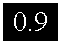 | 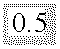 | 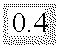 | 5 |
| 247 | 21148.4 | nucleobindin 2 | 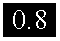 | 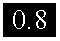 | 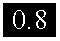 | 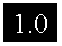 | 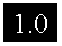 | 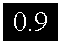 | 0.2 | 5 |
| 248 | 976749.1 | replication factor C (activator 1) 4 (37 kD) | 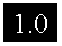 | 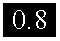 | 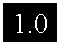 | 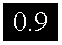 | 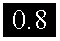 | 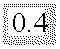 | 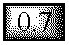 | 5 |
| 249 | 252719.12 | Human beta 3-endonexin mRNA, long form and short form, complete cds | 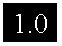 | 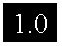 | 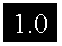 | 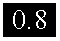 | 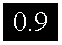 | 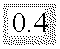 | 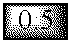 | 5 |
| 250 | g6063478 | G/T mismatch-binding protein | 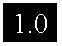 | 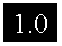 | 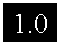 | 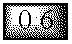 | 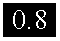 | 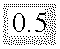 | 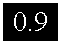 | 5 |
| 251 | 347314.3 | serine/threonine kinase 15 | 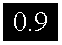 | 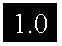 | 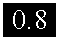 | 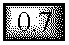 | 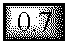 | 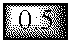 | 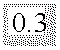 | 5 |
| 252 | g3213196 | serine/threonine kinase 15 | 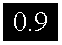 | 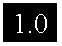 | 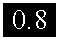 | 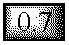 | 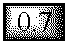 | 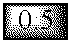 | 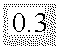 | 5 |
| 253 | 245184.3 | transforming growth factor, beta-induced 68 kD | 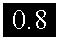 | 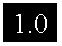 | 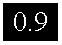 | 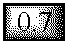 | 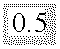 | 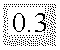 | 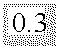 | 5 |
| 254 | 243574.11 | cysteine-rich protein 1 (intestinal) | 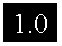 | 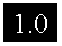 | 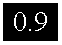 | 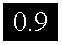 | 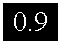 | 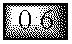 | 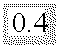 | 5 |
| 255 | 474826.6 | nidogen (enactin) | 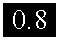 | 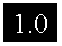 | 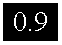 | 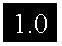 | 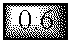 | 0.3 | 0.2 | 5 |
| 256 | 997347.6 | feline sarcoma viral (v-fes)/Fujinami avian sarcoma (PRCII) viral (v-fps) oncogene homolog | 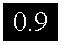 | 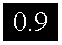 | 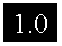 | 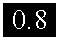 | 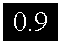 | 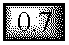 | 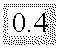 | 5 |
| 257 | 222049.1 | H. sapiens mRNA for glutamine cyclotransferase | 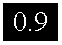 | 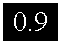 | 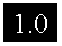 | 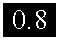 | 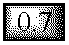 | 0.4 | 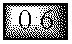 | 5 |
| 258 | 9902659.8 | small nuclear ribonucleoprotein polypeptide G | 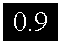 | 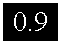 | 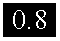 | 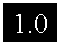 | 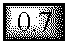 | 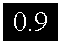 | 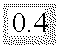 | 5 |
| 259 | 2508261 | interferon, gamma-inducible protein 16 | 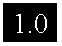 | 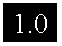 | 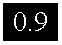 | 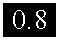 | 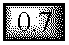 | 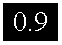 | 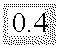 | 5 |
| 260 | 232945.12 | RAD54 (S. cerevisiae)-like | 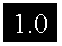 | 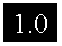 | 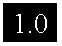 | 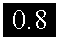 | 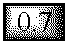 | 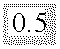 | 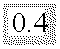 | 5 |
| 261 | 445101.8 | proliferating cell nuclear antigen | 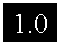 | 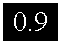 | 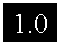 | 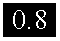 | 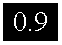 | 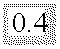 | 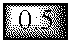 | 5 |
| 262 | 255750.1 | metallothionein 3 (growth inhibitory factor (neurotrophic)) | 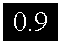 | 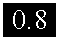 | 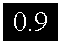 | 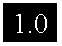 | 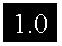 | 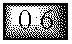 | 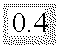 | 5 |
| 263 | 988231.7 | interferon-induced protein 17 | 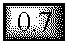 | 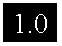 | 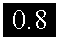 | 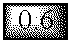 | 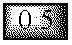 | 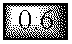 | 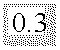 | 5 |
| 264 | 444902.6 | interferon-inducible | 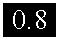 | 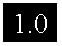 | 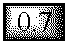 | 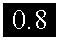 | 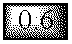 | 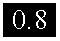 | 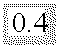 | 5 |
| 265 | 407546.8 | calreticulin | 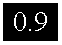 | 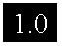 | 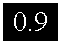 | 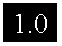 | 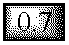 | 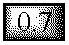 | 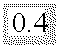 | 5 |
| 266 | 346511.4 | 2′-5′oligoadenylate synthetase 2 | 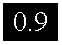 | 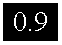 | 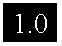 | 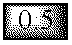 | 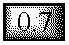 | 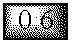 | 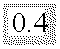 | 5 |
| 267 | 346411.5 | 2′-5′oligoadenylate synthetase 2 | 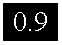 | 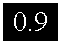 | 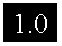 | 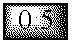 | 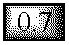 | 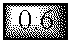 | 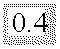 | 5 |
| 268 | 1098141.1 | breast cancer 1, early onset | 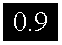 | 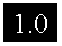 | 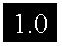 | 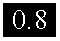 | 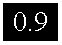 | 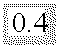 | 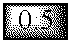 | 5 |
| 269 | 238089.2 | exonuclease 1 | 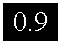 | 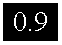 | 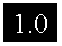 | 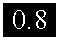 | 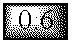 | 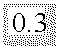 | 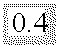 | 5 |
| 270 | 1100105.3 | CD74 antigen | 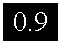 | 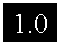 | 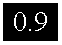 | 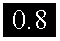 | 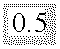 | 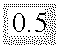 | 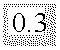 | 6 |
| 271 | 474729.2 | calponin 2 | 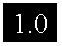 | 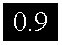 | 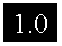 | 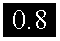 | 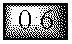 | 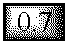 | 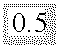 | 6 |
| 272 | 36300.3 | complement component 2 | 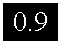 | 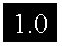 | 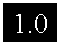 | 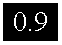 | 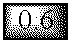 | 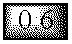 | 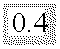 | 6 |
| 273 | 395096.3 | minichromosome maintenance deficient (S. cerevisiae) 2 (mitotine) | 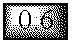 | 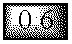 | 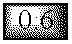 | 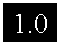 | 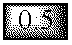 | 0.2 | 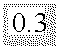 | 7 |
| 274 | 374086.1 | high-mobility group (nonhistone chromosomal) protein 1 | 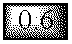 | 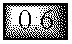 | 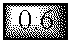 | 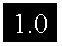 | 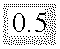 | 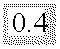 | 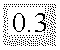 | 7 |
| 275 | 44495.4 | small nuclear ribonucleoprotein polypeptide F | 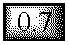 | 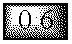 | 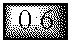 | 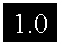 | 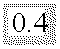 | 0.5 | 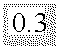 | 7 |
| 276 | 474876.2 | Human mRNA for Sm protein F | 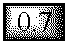 | 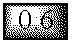 | 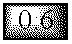 | 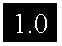 | 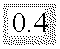 | 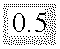 | 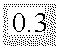 | 7 |

TABLE 2

| SEQ ID | | | | |
|---|---|---|---|---|
| NO | Incyte ID | Clone ID | Start | Stop |
| 1 | 440295.1 | 3034487 | 2203 | 3330 |
| 2 | g34387 | 79576 | 17 | 1395 |
| 3 | 247178.2 | 567292 | 3432 | 4661 |
| 4 | 567938 | 567938 | 669 | 1472 |
| 5 | 351122.2 | 682741 | 682 | 1151 |
| 6 | 481379.9 | 1219315 | 3730 | 4136 |
| 7 | 215391.7 | 1269046 | 861 | 1902 |
| 8 | 243812.1 | 1321761 | 698 | 1663 |
| 9 | 1085755.1 | 1376121 | 650 | 1291 |
| 10 | 347809.3 | 1516886 | 3615 | 4644 |
| 11 | 331734.4 | 1595081 | 334 | 876 |
| 12 | 116840.38 | 1606119 | 847 | 1284 |
| 13 | 903565.11 | 1672574 | 4016 | 4325 |
| 14 | 903565.8 | 1672574 | 1242 | 1787 |
| 15 | 474310.13 | 1672744 | 1281 | 3844 |
| 16 | 413006.13 | 1732479 | 1143 | 1904 |
| 17 | 76460.2 | 1749883 | 350 | 831 |
| 18 | 474374.4 | 2679117 | 1030 | 2542 |
| 19 | 427792.8 | 2806166 | 611 | 1994 |
| 20 | 364482.3 | 3178719 | 1331 | 1922 |
| 21 | 978487.1 | 3178719 | 54 | 526 |
| 22 | 410626.2 | 3602501 | 1153 | 1796 |
| 23 | 234480.6 | 1238577 | 298 | 1045 |
| 24 | 253542.2 | 1734561 | 1606 | 2355 |
| 25 | 234202.24 | 1995380 | 50 | 901 |
| 26 | 253946.4 | 2172334 | 1098 | 2397 |
| 27 | 348801.1 | 2203834 | 15 | 663 |
| 28 | 980611.1 | 2213735 | 1431 | 2249 |
| 29 | 283885.8 | 2415989 | 918 | 1576 |
| 30 | 348196.33 | 2852561 | 1095 | 1848 |
| 31 | 256009.4 | 3068454 | 4496 | 4936 |
| 32 | 481594.12 | 3211396 | 649 | 1098 |
| 33 | 978788.1 | 3211396 | 562 | 678 |
| 34 | 335171.1 | 3229778 | 5149 | 5670 |
| 35 | 998433.2 | 63038 | 2 | 1032 |
| 36 | 221928.9 | 674714 | 1199 | 1386 |
| 37 | 331291.3 | 1579487 | 3345 | 3833 |
| 38 | 233331.3 | 1712888 | 2259 | 2939 |
| 39 | 474682.2 | 1969044 | 1006 | 1509 |
| 40 | 3161.7 | 1484773 | 270 | 603 |
| 41 | 984248.1 | 1516047 | 968 | 1760 |
| 42 | 196590.2 | 1607510 | 723 | 1118 |
| 43 | 255109.1 | 1607510 | 304 | 429 |
| 44 | 238622.1 | 1669780 | 27 | 957 |
| 45 | 334385.3 | 1890138 | 6050 | 6479 |
| 46 | 998997.1 | 1640161 | 1104 | 1496 |
| 47 | 200578.1 | 1397926 | 1138 | 2288 |
| 48 | 208134.1 | 2293931 | 2596 | 2746 |
| 49 | 153659.2 | 519653 | 1355 | 1884 |
| 50 | 241930.15 | 1512213 | 1017 | 1540 |
| 51 | 413466.5 | 1985104 | 760 | 1861 |
| 52 | 3249239 | 3249239 | 740 | 2957 |
| 53 | 337518.18 | 3506985 | 151 | 500 |
| 54 | g3116213 | 2170638 | 194 | 1738 |
| 55 | g5912216 | 2170638 | 466 | 2010 |
| 56 | 992917.1 | 27775 | 386 | 910 |
| 57 | 411424.12 | 126888 | 88 | 599 |
| 58 | 995600.17 | 237730 | 799 | 1151 |
| 59 | 441292.7 | 1624024 | 1208 | 2738 |
| 60 | 42176.5 | 1650238 | 89 | 2297 |
| 61 | 234537.3 | 1718651 | 3061 | 3639 |
| 62 | 470468.21 | 1806435 | 800 | 1521 |
| 63 | 240120.3 | 1862257 | 472 | 2312 |
| 64 | 28779.3 | 2220923 | 8 | 785 |
| 65 | 238627.2 | 2555673 | 145 | 855 |
| 66 | 254107.1 | 2394637 | 3297 | 4186 |
| 67 | 330908.2 | 2987878 | 2395 | 3815 |
| 68 | g687589 | 1403041 | 170 | 1592 |
| 69 | 197975.11 | 1560143 | 2995 | 4347 |
| 70 | 227928.2 | 1719657 | 1717 | 2098 |
| 71 | 258785.7 | 1738168 | 3345 | 3738 |
| 72 | 977757.3 | 1830303 | 4598 | 7208 |
| 73 | 232773.2 | 1958631 | 2317 | 2963 |
| 74 | g6634024 | 2378601 | 697 | 1808 |
| 75 | g4589571 | 2902846 | 3036 | 3495 |
| 76 | 334370.3 | 3335055 | 1195 | 2483 |
| 77 | 980461.1 | 4003857 | 293 | 702 |
| 78 | 422969.4 | 1369536 | 3174 | 4219 |
| 79 | 244150.4 | 1429306 | 1803 | 5218 |
| 80 | 410257.11 | 1965978 | 2763 | 3546 |
| 81 | 28253.3 | 75549 | 425 | 661 |
| 82 | g31670 | 155892 | 1884 | 2388 |
| 83 | 977552.1 | 155904 | 993 | 1501 |
| 84 | 977552.2 | 155904 | 281 | 789 |
| 85 | 347829.6 | 185448 | 334 | 2046 |
| 86 | 251776.11 | 418731 | 2766 | 3414 |
| 87 | 343674.9 | 450618 | 919 | 1425 |
| 88 | 479136.1 | 885297 | 2474 | 3905 |
| 89 | 1078147.1 | 1000508 | 1289 | 2523 |
| 90 | 474275.1 | 1297562 | 4431 | 5815 |
| 91 | g403532 | 1320658 | 1213 | 2771 |
| 92 | 242114.16 | 1361963 | 2792 | 4530 |
| 93 | 445186.7 | 1375107 | 243 | 1602 |
| 94 | 474496.2 | 1401002 | 1855 | 2387 |
| 95 | 257114.7 | 1424573 | 867 | 1703 |
| 96 | 984005.1 | 1446475 | 68 | 809 |
| 97 | 977667.1 | 1447909 | 1146 | 1705 |
| 98 | 996862.4 | 1449337 | 50 | 660 |
| 99 | 364940.19 | 1479437 | 2522 | 5308 |
| 100 | 1041140.4 | 2220025 | 448 | 2428 |
| 101 | 408246.2 | 1595756 | 890 | 1796 |
| 102 | 902740.4 | 1670773 | 373 | 845 |
| 103 | 475486.9 | 1694039 | 483 | 1534 |
| 104 | 233778.9 | 1695477 | 970 | 1500 |
| 105 | 350392.3 | 1719058 | 2417 | 4573 |
| 106 | 458045.4 | 1720114 | 2424 | 4196 |
| 107 | 471362.17 | 1720149 | 286 | 1089 |
| 108 | 336716.3 | 1749727 | 1431 | 2412 |
| 109 | 995211.5 | 1782172 | 1192 | 3936 |
| 110 | 238824.2 | 1841989 | 1082 | 2360 |
| 111 | 474592.3 | 1846463 | 2393 | 3281 |
| 112 | 431338.2 | 1890243 | 876 | 2359 |
| 113 | 412631.5 | 1907232 | 12440 | 12947 |
| 114 | 350480.6 | 1975575 | 1928 | 2274 |
| 115 | 350521.15 | 2078364 | 1075 | 1890 |
| 116 | 445076.9 | 2449986 | 356 | 1578 |
| 117 | 995028.4 | 2483605 | 132 | 606 |
| 118 | 245008.4 | 2900572 | 2225 | 3836 |
| 119 | 350895.1 | 2952864 | 440 | 1439 |
| 120 | 434265.5 | 3421442 | 740 | 1203 |
| 121 | 427813.14 | 3553729 | 6501 | 7091 |
| 122 | 14704.3 | 3742428 | 1000 | 2154 |
| 123 | 344240.2 | 3943651 | 2050 | 2530 |
| 124 | 239694.6 | 4144156 | 2287 | 3032 |
| 125 | 255772.2 | 433573 | 1321 | 2758 |
| 126 | 232066.3 | 514726 | 2248 | 2778 |
| 127 | 246504.1 | 570512 | 750 | 2361 |
| 128 | 986123.22 | 1522716 | 1264 | 1904 |
| 129 | 898945.14 | 1525829 | 830 | 1628 |
| 130 | 236208.16 | 1682642 | 2775 | 3010 |
| 131 | 246531.2 | 1692164 | 1374 | 1602 |
| 132 | 238586.2 | 1699587 | 427 | 910 |
| 133 | 245532.7 | 1804548 | 1196 | 1992 |
| 134 | 200972.2 | 1850135 | 1308 | 2138 |
| 135 | 348061.1 | 1867652 | 687 | 2825 |
| 136 | 233711.7 | 1902929 | 1146 | 2151 |
| 137 | 256043.19 | 1910469 | 1137 | 1625 |
| 138 | 445012.6 | 1911016 | 6908 | 7424 |
| 139 | g463906 | 1959969 | 29 | 523 |
| 140 | 475621.1 | 1967160 | 1459 | 1932 |
| 141 | 216063.17 | 2174920 | 381 | 1030 |
| 142 | 1099498.9 | 2369312 | 463 | 601 |
| 143 | 1099076.1 | 2537805 | 299 | 664 |
| 144 | 902119.3 | 2594308 | 1 | 836 |
| 145 | g2982500 | 2720693 | 3244 | 4316 |
| 146 | 1097580.4 | 2733928 | 126 | 1118 |
| 147 | 391851.1 | 2868138 | 490 | 851 |
| 148 | 13105.9 | 2921194 | 462 | 1362 |
| 149 | 356248.4 | 2967860 | 1184 | 5905 |
| 150 | 331045.1 | 3001809 | 2899 | 4165 |
| 151 | 482480.3 | 3003077 | 2356 | 2814 |
| 152 | 245099.8 | 3119252 | 1681 | 2281 |
| 153 | 245481.2 | 3606947 | 243 | 1980 |
| 154 | 225021.4 | 146667 | 1489 | 2773 |
| 155 | 451767.28 | 418041 | 66 | 864 |
| 156 | 902142.11 | 518094 | 1155 | 1925 |
| 157 | 291095.5 | 719318 | 4551 | 5099 |
| 158 | 332919.4 | 924319 | 781 | 1262 |
| 159 | 387130.26 | 1439677 | 133 | 3079 |
| 160 | 410580.13 | 1445767 | 645 | 2172 |
| 161 | 251715.1 | 1705208 | 1702 | 2383 |
| 162 | 1799017F6 | 1799017 | 1 | 459 |
| 163 | 348891.1 | 1877829 | 777 | 1288 |
| 164 | 903956.15 | 1879023 | 1328 | 3314 |
| 165 | 235184.1 | 1988432 | 660 | 979 |
| 166 | 330948.3 | 2054252 | 3800 | 4487 |
| 167 | 994057.1 | 2055534 | 4841 | 5856 |
| 168 | 197301.4 | 2591814 | 937 | 3287 |
| 169 | 476016.17 | 3142624 | 419 | 1641 |
| 170 | 1098409.1 | 3603037 | 1338 | 2945 |
| 171 | 202023.6 | 160822 | 2991 | 4412 |
| 172 | 350423.5 | 1624459 | 56 | 1711 |
| 173 | 1100023.1 | 2895245 | 512 | 2019 |
| 174 | 414196.8 | 1222317 | 1 | 476 |
| 175 | 331106.6 | 1518328 | 1675 | 2011 |
| 176 | g180670 | 1558081 | 1074 | 2596 |
| 177 | 236574.12 | 1559730 | 2628 | 3663 |
| 178 | 1000033.6 | 1600726 | 1229 | 4571 |
| 179 | 37567.22 | 1672930 | 185 | 829 |
| 180 | 995610.1 | 1673876 | 1688 | 2958 |
| 181 | 1702374 | 1702374 | 1038 | 3139 |
| 182 | 427883.47 | 1881243 | −14 | 435 |
| 183 | 93687.6 | 1907952 | 1259 | 1638 |
| 184 | 414100.4 | 1931275 | 323 | 1871 |
| 185 | 235148.4 | 1987127 | 199 | 873 |
| 186 | 430039.3 | 1988710 | 932 | 1388 |
| 187 | 348110.2 | 2158373 | 2064 | 2281 |
| 188 | 1098815.7 | 2831248 | 393 | 924 |
| 189 | 474491.18 | 3747901 | 190 | 1319 |
| 190 | 474491.19 | 3747901 | 267 | 1396 |
| 191 | 419031.5 | 1988019 | 1627 | 2079 |
| 192 | 399658.1 | 3967402 | 559 | 1697 |
| 193 | 474913.3 | 3138128 | 3685 | 4570 |
| 194 | 199898.3 | 1217764 | 271 | 932 |
| 195 | 253550.14 | 1447903 | 441 | 2458 |
| 196 | 331597.2 | 1975944 | 3277 | 4284 |
| 197 | 997377.1 | 1526665 | 256 | 788 |
| 198 | 42869.3 | 2016960 | 162 | 835 |
| 199 | 248306.1 | 2474163 | 198 | 1710 |
| 200 | 247220.15 | 39817 | 846 | 1550 |
| 201 | 26662.3 | 485111 | 7807 | 10242 |
| 202 | 977509.3 | 494905 | 166 | 2605 |
| 203 | 221961.2 | 633460 | 945 | 1672 |
| 204 | 246824.1 | 1488852 | 319 | 789 |
| 205 | 407557.2 | 1501556 | 1391 | 2055 |
| 206 | 372981.2 | 1576329 | 551 | 906 |
| 207 | 201409.6 | 1622987 | 791 | 1342 |
| 208 | 331025.1 | 2242674 | 1364 | 2791 |
| 209 | 247515.1 | 2399253 | 606 | 1044 |
| 210 | 199471.2 | 2414624 | 125 | 1464 |
| 211 | 2916753 | 2916753 | 114 | 1109 |
| 212 | 343899.2 | 3622417 | 97 | 897 |
| 213 | 335775.2 | 3771476 | 1324 | 2846 |
| 214 | 232714.5 | 277897 | 479 | 649 |
| 215 | 305039.4 | 522991 | 1009 | 1391 |
| 216 | 233603.2 | 1604056 | 1 | 190 |
| 217 | 330930.1 | 1740384 | 6110 | 6515 |
| 218 | 247289.1 | 1901271 | 2050 | 2558 |
| 219 | 331033.1 | 1970111 | 1079 | 2827 |
| 220 | 1098766.1 | 2113618 | 939 | 1345 |
| 221 | 245632.3 | 2396287 | 2506 | 2827 |
| 222 | 333461.2 | 4003342 | 1541 | 2067 |
| 223 | 347876.6 | 103669 | 290 | 2971 |
| 224 | 413842.1 | 173591 | 1 | 366 |
| 225 | 235867.2 | 343653 | 1519 | 2159 |
| 226 | 199636.2 | 428665 | 800 | 2432 |
| 227 | 2234.3 | 627654 | 124 | 682 |
| 228 | 1000139.13 | 690313 | 557 | 1118 |
| 229 | 998534.1 | 885129 | 1823 | 2762 |
| 230 | 372377.6 | 1315115 | 2445 | 2827 |
| 231 | 1101412.4 | 1340504 | 440 | 938 |
| 232 | 261567.5 | 1384823 | 32 | 576 |
| 233 | 232713.2 | 1405652 | 436 | 2078 |
| 234 | 214335.13 | 1439126 | 904 | 2629 |
| 235 | 331022.33 | 1485479 | 3584 | 4373 |
| 236 | 332259.3 | 1513664 | 2446 | 3297 |
| 237 | 253570.8 | 1516301 | 467 | 1240 |
| 238 | 995529.5 | 1525795 | 337 | 1781 |
| 239 | 474435.16 | 1610523 | 1323 | 2032 |
| 240 | 994861.1 | 1623237 | 9447 | 10862 |
| 241 | g545708 | 1668794 | 39 | 798 |
| 242 | 347965.2 | 1672749 | 734 | 1895 |
| 243 | 202361.1 | 1700047 | 1121 | 1558 |
| 244 | 369950.12 | 1702350 | 840 | 1323 |
| 245 | 331403.8 | 1746529 | 2145 | 2537 |
| 246 | 233889.3 | 1758241 | 687 | 941 |
| 247 | 21148.4 | 1760517 | 462 | 1583 |
| 248 | 976749.1 | 1773638 | 1907 | 2394 |
| 249 | 252719.12 | 1809385 | 61 | 1008 |
| 250 | g6063478 | 1926006 | 3426 | 4214 |
| 251 | 347314.3 | 2007691 | 1146 | 2115 |
| 252 | g3213196 | 2007691 | 1240 | 2209 |
| 253 | 245184.3 | 2056395 | 946 | 2668 |
| 254 | 243574.11 | 2121863 | 581 | 836 |
| 255 | 474826.6 | 2175008 | 4211 | 4706 |
| 256 | 997347.6 | 2195430 | 2222 | 2834 |
| 257 | 222049.1 | 2365295 | 62 | 588 |
| 258 | 902659.8 | 2449837 | 403 | 806 |
| 259 | 2508261 | 2508261 | 541 | 2671 |
| 260 | 232945.12 | 2645840 | 968 | 2501 |
| 261 | 445101.8 | 2781405 | 911 | 1316 |
| 262 | 255750.1 | 2901811 | 129 | 471 |
| 263 | 988231.7 | 2902903 | 435 | 1058 |
| 264 | 444902.6 | 2949427 | 92 | 664 |
| 265 | 407546.8 | 2970280 | 564 | 1888 |
| 266 | 346511.4 | 3214930 | 17 | 588 |
| 267 | 346511.5 | 3214930 | 81 | 652 |
| 268 | 1098141.1 | 3563535 | 4062 | 4478 |
| 269 | 238089.2 | 4385292 | 1184 | 3103 |
| 270 | 1100105.3 | 1001730 | 91 | 1412 |
| 271 | 474729.2 | 1443061 | 319 | 2116 |
| 272 | 363000.3 | 1510424 | 965 | 2927 |
| 273 | 395096.3 | 1723834 | 2914 | 3253 |
| 274 | 374086.1 | 1813133 | 89 | 862 |
| 275 | 444495.4 | 2104530 | 743 | 1309 |
| 276 | 474876.2 | 2104530 | 148 | 472 |

TABLE 3

| | | | | | | | | | | | | |
|---|---|---|---|---|---|---|---|---|---|---|---|---|
| SEQ | | | | | | | | | | | | |
| ID | | | | | | | | | | max | max | max |
| NO | Incyte ID | Gene Annotation | 0h | 0.5h | 2.5h | 8h | 1d | 2d | 4d | up | down | diff |

| | | | | | | | | | | | | | | |
|---|---|---|---|---|---|---|---|---|---|---|---|---|---|---|
| 1 | 3034487 | solute carrier family 4, sodium bicarbonate | 1.00 | 0.96 | 1.15 | 2.02 | 1.20 | 1.45 | 3.51 | 3.51 | 0.96 | 3.51 | up- | |
| | | cotransporter, member 6 | | | | | | | | | | | regu- | |
| | | | | | | | | | | | | | | lated |
| 2 | g34387 | annexin I (lipocortin I) | 1.00 | 1.23 | 1.33 | 1.23 | 2.83 | 2.54 | 1.86 | 2.83 | 1.00 | 2.83 | | |
| 3 | 247178.2 | sperm surface protein | 1.00 | 0.99 | 1.11 | 1.24 | 2.00 | 1.93 | 2.00 | 2.00 | 0.99 | 2.00 | | |
| 4 | 567938 | integrin, alpha X (antigen CD11C (p150), | 1.00 | 1.03 | 1.05 | 0.95 | 2.11 | 1.53 | 2.45 | 2.45 | 0.95 | 2.45 | | |
| | | alpha polypeptide) | | | | | | | | | | | | |
| 5 | 351122.2 | integrin, beta 3 (platelet glycoprotein | 1.00 | 1.05 | 1.07 | 0.87 | 3.20 | 2.22 | 2.61 | 3.20 | 0.87 | 3.20 | | |
| | | IIIa, antigen CD61) | | | | | | | | | | | | |
| 6 | 481379.9 | paired basic amino acid cleaving enzyme | 1.00 | 1.08 | 1.01 | 1.03 | 2.06 | 1.99 | 1.57 | 2.06 | 1.00 | 2.06 | | |
| | | (furin, membrane associated receptor protein) | | | | | | | | | | | | |
| 7 | 215391.7 | phosphogluconate dehydrogenase | 1.00 | 1.47 | 1.24 | 1.78 | 2.59 | 2.49 | 2.54 | 2.59 | 1.00 | 2.59 | | |
| 8 | 243812.1 | protein kinase mitogen- activated 13 | 1.00 | 1.17 | 1.19 | 0.96 | 2.27 | 1.91 | 2.68 | 2.68 | 0.96 | 2.68 | | |
| 9 | 1085755.1 | folate receptor 1 (adult) | 1.00 | 0.80 | 1.00 | 1.07 | 2.28 | 1.89 | 2.16 | 2.28 | 0.80 | 2.28 | | |
| 10 | 347809.3 | solute carrier family 6 (neurotransmitter | 1.00 | 1.06 | 1.33 | 1.21 | 2.65 | 2.13 | 3.06 | 3.06 | 1.00 | 3.06 | | |
| | | transporter, taurine), member 6 | | | | | | | | | | | | |
| 11 | 331734.4 | prostaglandin-endoperoxide synthase 1 | 1.00 | 1.20 | 1.17 | 1.30 | 2.08 | 1.50 | 1.72 | 2.08 | 1.00 | 2.08 | | |
| | | (prostaglandin G/H synthase and cyclooxygena | | | | | | | | | | | | |
| 12 | 116840.38 | interferon regulatory factor 3 | 1.00 | 1.15 | 1.02 | 0.94 | 2.31 | 2.37 | 1.85 | 2.37 | 0.94 | 2.37 | | |
| 13 | 903565.11 | proprotein convertase subtilisin/kexin type 4 | 1.00 | 1.11 | 1.18 | 1.13 | 2.12 | 1.68 | 2.50 | 2.50 | 1.00 | 2.50 | | |
| 14 | 903565.8 | Human mRNA for PACE4E-I, complete cds. | 1.00 | 1.11 | 1.18 | 1.13 | 2.12 | 1.68 | 2.50 | 2.50 | 1.00 | 2.50 | | |
| 15 | 474310.13 | transglutaminase 2 (C polypeptide, | 1.00 | 1.20 | 1.09 | 0.78 | 2.17 | 3.20 | 3.29 | 3.29 | 0.78 | 3.29 | | |
| | | protein-glutamine-gamma-glutamyltransferase) | | | | | | | | | | | | |
| 16 | 413006.13 | differentiated Embryo Chondrocyte | 1.00 | 1.17 | 1.03 | 1.07 | 2.16 | 2.25 | 1.59 | 2.25 | 1.00 | 2.25 | | |
| | | expressed gene 1 | | | | | | | | | | | | |
| 17 | 76460.2 | pyridoxal (pyridoxine, vitamin B6) kinase | 1.00 | 1.23 | 1.16 | 1.54 | 2.51 | 1.61 | 1.90 | 2.51 | 1.00 | 2.51 | | |
| 18 | 474374.4 | pim-1 oncogene | 1.00 | 1.16 | 1.09 | 1.13 | 3.56 | 2.75 | 3.02 | 3.56 | 1.00 | 3.56 | | |
| 19 | 427792.8 | cathepsin B | 1.00 | 1.25 | 1.01 | 1.51 | 2.13 | 1.78 | 1.37 | 2.13 | 1.00 | 2.13 | | |
| 20 | 364482.3 | carnitine palmitoyltransferase I, liver | 1.00 | 0.81 | 0.98 | 1.25 | 2.08 | 2.06 | 1.61 | 2.08 | 0.81 | 2.08 | | |
| 21 | 978487.1 | carnitine palmitoyltransferase I, liver | 1.00 | 0.81 | 0.98 | 1.25 | 2.08 | 2.06 | 1.61 | 2.08 | 0.81 | 2.08 | | |
| 22 | 410626.2 | Human retinoid X receptor-gamma | 1.00 | 1.08 | 1.19 | 1.66 | 2.48 | 2.51 | 3.09 | 3.09 | 1.00 | 3.09 | | |
| | | mRNA, complete cds | | | | | | | | | | | | |
| 23 | 234480.6 | glutaredoxin (thioltransferase) | 1.00 | 0.91 | 0.91 | 0.78 | 3.65 | 3.13 | 1.08 | 3.65 | 0.78 | 3.65 | | |
| 24 | 253542.2 | dual specificity phosphatase 5 | 1.00 | 0.92 | 0.84 | 1.01 | 3.83 | 3.38 | 2.23 | 3.83 | 0.84 | 3.83 | | |
| 25 | 234202.24 | microsomal glutathione S-transferase 1 | 1.00 | 0.93 | 1.02 | 1.28 | 3.07 | 2.33 | 1.68 | 3.07 | 0.93 | 3.07 | | |
| 26 | 253946.4 | interleukin 6 signal transducer | 1.00 | 1.01 | 1.29 | 1.07 | 2.04 | 0.94 | 2.24 | 2.24 | 0.94 | 2.24 | | |
| | | (gp130, oncostatin M receptor) | | | | | | | | | | | | |
| 27 | 348801.1 | pro-platelet basic protein | 1.00 | 1.01 | 1.03 | 1.00 | 3.77 | 3.69 | 1.81 | 3.77 | 1.00 | 3.77 | | |
| 28 | 980611.1 | matrilin 1, cartilage matrix protein | 1.00 | 0.94 | 0.92 | 0.90 | 2.86 | 0.73 | 1.25 | 2.86 | 0.73 | 2.86 | | |
| 29 | 283885.8 | dual-specificity tyrosine-(Y)-phosphorylation | 1.00 | 1.13 | 1.08 | 0.88 | 2.64 | 1.06 | 1.75 | 2.64 | 0.88 | 2.64 | | |
| | | regulated kinase 4 | | | | | | | | | | | | |
| 30 | 348196.33 | antigen identified by monoclonal antibodies | 1.00 | 1.14 | 0.98 | 1.35 | 2.96 | 2.07 | 1.64 | 2.96 | 0.98 | 2.96 | | |
| | | 4F2, TRA1.10, TROP4, and T43 | | | | | | | | | | | | |
| 31 | 256009.4 | AHNAK nucleoprotein (desmoyokin) | 1.00 | 1.17 | 1.33 | 0.98 | 2.32 | 2.35 | 0.98 | 2.35 | 0.98 | 2.35 | | |
| 32 | 481594.12 | Human RACH1 (RACH1) mRNA, | 1.00 | 1.24 | 1.27 | 0.72 | 2.59 | 0.79 | 0.79 | 2.59 | 0.72 | 2.59 | | |
| | | complete cds | | | | | | | | | | | | |
| 33 | 978788.1 | Human RACH1 (RACH1) mRNA, | 1.00 | 1.24 | 1.27 | 0.72 | 2.59 | 0.79 | 0.79 | 2.59 | 0.72 | 2.59 | | |
| | | complete cds | | | | | | | | | | | | |
| 34 | 335171.1 | integrin, alpha 2 (CD49B, alpha 2 | 1.00 | 1.13 | 1.16 | 0.68 | 2.13 | 1.10 | 2.34 | 2.34 | 0.68 | 2.34 | | |
| | | subunit of VLA-2 receptor) | | | | | | | | | | | | |
| 35 | 998433.2 | ESTs, Highly similar to DIAMINE | 1.00 | 1.22 | 1.15 | 1.38 | 2.81 | 2.41 | 2.27 | 2.81 | 1.00 | 2.81 | | |
| | | ACETYLTRANSFERASE [H. sapiens] | | | | | | | | | | | | |
| 36 | 221928.9 | ESTs | 1.00 | 0.94 | 1.02 | 1.09 | 3.25 | 2.26 | 2.72 | 3.25 | 0.94 | 3.25 | | |
| 37 | 331291.3 | Homo sapiens mRNA for KIAA0291 | 1.00 | 1.26 | 1.17 | 1.15 | 2.42 | 1.44 | 1.65 | 2.42 | 1.00 | 2.42 | | |
| | | gene, partial cds | | | | | | | | | | | | |
| 38 | 233331.3 | Homo sapiens KIAA0439 mRNA, partial cds | 1.00 | 1.18 | 1.11 | 0.80 | 2.12 | 2.75 | 2.47 | 2.75 | 0.80 | 2.75 | | |
| 39 | 474682.2 | ESTs, Weakly similar to W01A11.2 | 1.00 | 1.17 | 1.34 | 1.27 | 3.61 | 2.29 | 3.71 | 3.71 | 1.00 | 3.71 | | |
| | | gene product [C.elegans] | | | | | | | | | | | | |
| 40 | 3161.7 | ESTs, Weakly similar to (defline not | 1.00 | 1.06 | 1.02 | 0.84 | 2.84 | 1.13 | 1.03 | 2.84 | 0.84 | 2.84 | | |
| | | available 4529890) [H. sapiens] | | | | | | | | | | | | |
| 41 | 984248.1 | ESTs | 1.00 | 1.02 | 1.01 | 0.73 | 2.14 | 0.75 | 1.37 | 2.14 | 0.73 | 2.14 | | |
| 42 | 196590.2 | ESTs | 1.00 | 1.12 | 1.08 | 0.76 | 2.41 | 0.82 | 0.75 | 2.41 | 0.75 | 2.41 | | |
| 43 | 255109.1 | ESTs | 1.00 | 1.12 | 1.08 | 0.76 | 2.41 | 0.82 | 0.75 | 2.41 | 0.75 | 2.41 | | |
| 44 | 238622.1 | Human clone 46690 brain expressed | 1.00 | 1.06 | 0.93 | 0.77 | 2.11 | 0.95 | 2.04 | 2.11 | 0.77 | 2.11 | | |
| | | mRNA from chromosome X | | | | | | | | | | | | |
| 45 | 334385.3 | Homo sapiens mRNA for KIAA0284 | 1.00 | 1.20 | 1.13 | 0.87 | 2.00 | 0.83 | 1.39 | 2.00 | 0.83 | 2.00 | | |
| | | gene, partial cds | | | | | | | | | | | | |
| 46 | 998997.1 | ESTs | 1.00 | 1.05 | 1.01 | 2.37 | 0.84 | 0.93 | 1.18 | 2.37 | 0.84 | 2.37 | | |
| 47 | 200578.1 | ESTs | 1.00 | 1.18 | 1.25 | 1.75 | 5.54 | 5.02 | 3.11 | 5.54 | 1.00 | 5.54 | | |
| 48 | 208134.1 | ESTs | 1.00 | 1.17 | 1.09 | 0.81 | 3.53 | 2.33 | 4.59 | 4.59 | 0.81 | 4.59 | | |
| 49 | 153659.2 | interleukin 1 receptor antagonist | 1.00 | 1.33 | 1.29 | 1.26 | 2.00 | 3.16 | 4.88 | 4.88 | 1.00 | 4.88 | | |
| 50 | 241930.15 | liver X receptor, alpha | 1.00 | 1.13 | 1.28 | 2.29 | 2.15 | 2.27 | 4.34 | 4.34 | 1.00 | 4.34 | | |
| 51 | 413466.5 | adipose differentiation-related | 1.00 | 1.62 | 2.66 | 4.30 | 7.11 | 7.12 | 14.12 | 14.12 | 1.00 | 14.12 | | |
| | | protein; adipophilin | | | | | | | | | | | | |
| 52 | 3249239 | colony stimulating factor 1 (macrophage) | 1.00 | 1.20 | 1.18 | 1.24 | 2.42 | 2.41 | 4.73 | 4.73 | 1.00 | 4.73 | | |
| 53 | 337518.18 | CD36 antigen (collagen type I receptor, | 1.00 | 1.19 | 1.39 | 2.00 | 3.49 | 2.68 | 4.32 | 4.32 | 1.00 | 4.32 | | |
| | | thrombospondin receptor) | | | | | | | | | | | | |
| 54 | g3116213 | SH3 binding protein | 1.00 | 1.25 | 1.10 | 1.50 | 4.20 | 3.19 | 2.44 | 4.20 | 1.00 | 4.20 | | |
| 55 | g5912216 | SH3 binding protein | 1.00 | 1.25 | 1.10 | 1.50 | 4.20 | 3.19 | 2.44 | 4.20 | 1.00 | 4.20 | | |
| 171 | 202023.6 | 3-hydroxy-3-methylglutaryl-Coenzyme | 1.00 | 0.96 | 0.87 | 0.50 | 0.52 | 0.68 | 0.61 | 1.00 | 0.50 | 2.01 | down- | |
| | | A reductase | | | | | | | | | | | regu- | |
| | | | | | | | | | | | | | lated | |
| 172 | 350423.5 | farnesyl-diphosphate farnesyltransferase 1 | 1.00 | 1.19 | 1.05 | 0.49 | 0.47 | 0.55 | 0.54 | 1.19 | 0.47 | 2.14 | | |
| 173 | 1100023.1 | cytochrome P450, 51 (lanosterol | 1.00 | 1.31 | 1.33 | 0.62 | 0.41 | 0.59 | 0.70 | 1.33 | 0.41 | 2.44 | | |
| | | 14-alpha-demethylase) | | | | | | | | | | | | |
| 174 | 414196.8 | S100 calcium-binding protein A4 | 1.00 | 1.06 | 1.06 | 0.97 | 0.34 | 0.61 | 0.41 | 1.06 | 0.34 | 2.91 | | |
| 175 | 331106.6 | integrin, alpha 6 | 1.00 | 0.94 | 1.05 | 1.08 | 0.45 | 0.76 | 0.81 | 1.08 | 0.45 | 2.23 | | |
| 176 | g180670 | matrix metalloproteinase 2 (gelatinase A, 72 | 1.00 | 1.22 | 1.13 | 0.92 | 0.48 | 0.72 | 0.67 | 1.22 | 0.48 | 2.10 | | |
| | | kD gelatinase, 72 kD type IV collagenase) | | | | | | | | | | | | |
| 177 | 236574.12 | macrophage-associated antigen | 1.00 | 1.17 | 1.20 | 0.95 | 0.40 | 0.54 | 1.04 | 1.20 | 0.40 | 2.48 | | |
| 178 | 1000033.6 | alpha-2-macroglobulin | 1.00 | 1.09 | 1.01 | 0.85 | 0.39 | 0.85 | 1.03 | 1.09 | 0.39 | 2.57 | | |
| 179 | 37567.22 | RAN binding protein 1 | 1.00 | 0.90 | 0.98 | 0.99 | 0.49 | 0.56 | 0.52 | 1.00 | 0.49 | 2.04 | | |
| 180 | 995610.1 | v-myc avian myelocytomatosis | 1.00 | 1.17 | 1.16 | 0.91 | 0.46 | 0.81 | 0.86 | 1.17 | 0.46 | 2.19 | | |
| | | viral oncogene homolog | | | | | | | | | | | | |
| 181 | 1702374 | v-myc avian myelocytomatosis viral oncogene | 1.00 | 1.11 | 1.08 | 0.81 | 0.49 | 0.77 | 0.58 | 1.11 | 0.49 | 2.03 | | |
| | | homolog 1, lung carcinoma derived | | | | | | | | | | | | |
| 182 | 427883.47 | Homo sapiens LST1 mRNA, cLST1/E | 1.00 | 1.15 | 0.98 | 1.00 | 0.40 | 0.76 | 0.45 | 1.15 | 0.40 | 2.49 | | |
| | | splice variant, complete cds | | | | | | | | | | | | |
| 183 | 93687.6 | uncoupling protein 2 (mitochondrial, | 1.00 | 1.09 | 1.12 | 1.05 | 0.50 | 0.51 | 0.46 | 1.12 | 0.46 | 2.18 | | |
| | | proton carrier) | | | | | | | | | | | | |
| 184 | 414100.4 | leukocyte-associated Ig-like receptor 1 | 1.00 | 1.09 | 1.10 | 1.26 | 0.40 | 0.49 | 0.51 | 1.26 | 0.40 | 2.47 | | |
| 185 | 235148.4 | arachidonate 5-lipoxygenase-activating protein | 1.00 | 0.96 | 0.96 | 0.98 | 0.30 | 0.48 | 0.86 | 1.00 | 0.30 | 3.39 | | |
| 186 | 430039.3 | CD14 antigen | 1.00 | 1.17 | 1.10 | 0.99 | 0.48 | 0.63 | 1.10 | 1.17 | 0.48 | 2.07 | | |
| 187 | 2158373F6 | platelet-derived growth factor | 1.00 | 1.03 | 0.97 | 1.05 | 0.49 | 1.00 | 1.46 | 1.46 | 0.49 | 2.04 | | |
| | | alpha polypeptide | | | | | | | | | | | | |
| 188 | 1098815.7 | Not mapped | 1.00 | 0.93 | 0.96 | 1.13 | 0.50 | 0.78 | 0.53 | 1.13 | 0.50 | 2.00 | | |
| 189 | 474491.18 | Human apurinic/apyrimidinic endonuclease | 1.00 | 1.18 | 1.13 | 1.28 | 0.48 | 0.83 | 0.76 | 1.28 | 0.48 | 2.09 | | |
| | | mRNA, complete cds. | | | | | | | | | | | | |
| 190 | 474491.19 | ref-1 | 1.00 | 1.18 | 1.13 | 1.28 | 0.48 | 0.83 | 0.76 | 1.28 | 0.48 | 2.09 | | |
| 191 | 419031.5 | leukotriene A4 hydrolase | 1.00 | 1.19 | 0.95 | 1.40 | 0.35 | 0.63 | 0.64 | 1.40 | 0.35 | 2.84 | | |
| 192 | 399658.1 | Not mapped | 1.00 | 1.11 | 1.00 | 0.77 | 0.46 | 0.53 | 1.47 | 1.47 | 0.46 | 2.18 | | |
| 193 | 474913.3 | ESTs | 1.00 | 1.01 | 0.98 | 0.77 | 0.35 | 1.00 | 0.81 | 1.01 | 0.35 | 2.84 | | |
| 194 | 199898.3 | Human G0S2 protein gene, complete cds | 1.00 | 1.07 | 1.10 | 0.87 | 0.21 | 0.41 | 0.56 | 1.10 | 0.21 | 4.84 | | |
| 195 | 253550.14 | insulin-like growth factor binding protein 3 | 1.00 | 1.06 | 1.06 | 1.37 | 0.28 | 0.90 | 1.18 | 1.37 | 0.28 | 3.62 | | |
| 196 | 331597.2 | cytochrome b-245, beta polypeptide | 1.00 | 1.14 | 1.08 | 1.02 | 0.25 | 0.28 | 0.16 | 1.14 | 0.16 | 6.32 | | |
| | | (chronic granulomatous disease) | | | | | | | | | | | | |

TABLE 4

| SEQ | | | | | | | | | | | | | |
|---|---|---|---|---|---|---|---|---|---|---|---|---|---|
| ID | | | | | | | | | | max | max | max | |
| NO | Incyte ID | Gene Annotation | 0h | 0.5h | 2.5h | 8h | 1d | 2d | 4d | up | down | diff | |
| 47 | 200578.1 | ESTs | 1.00 | 1.18 | 1.25 | 1.75 | 5.54 | 5.02 | 3.11 | 5.54 | 1.00 | 5.54 | up- |
| | | | | | | | | | | | | | regu- |
| | | | | | | | | | | | | | lated |
| 48 | 208134.1 | ESTs | 1.00 | 1.17 | 1.09 | 0.81 | 3.53 | 2.33 | 4.59 | 4.59 | 0.81 | 4.59 | |
| 49 | 153659.2 | interleukin 1 receptor antagonist | 1.00 | 1.33 | 1.29 | 1.26 | 2.00 | 3.16 | 4.88 | 4.88 | 1.00 | 4.88 | |
| 50 | 241930.15 | liver X receptor, alpha | 1.00 | 1.13 | 1.28 | 2.29 | 2.15 | 2.27 | 4.34 | 4.34 | 1.00 | 4.34 | |
| 51 | 413466.5 | adipose differentiation-related | 1.00 | 1.62 | 2.66 | 4.30 | 7.11 | 7.12 | 14.12 | 14.12 | 1.00 | 14.12 | |
| | | protein; adipophilin | | | | | | | | | | | |
| 52 | 3249239 | colony stimulating factor 1 (macrophage) | 1.00 | 1.20 | 1.18 | 1.24 | 2.42 | 2.41 | 4.73 | 4.73 | 1.00 | 4.73 | |
| 53 | 337518.18 | CD36 antigen (collagen type I | 1.00 | 1.19 | 1.39 | 2.00 | 3.49 | 2.68 | 4.32 | 4.32 | 1.00 | 4.32 | |
| | | receptor, thrombospondin receptor) | | | | | | | | | | | |
| 54 | g3116213 | SH3 binding protein | 1.00 | 1.25 | 1.10 | 1.50 | 4.20 | 3.19 | 2.44 | 4.20 | 1.00 | 4.20 | |
| 55 | g5912216 | SH3 binding protein | 1.00 | 1.25 | 1.10 | 1.50 | 4.20 | 3.19 | 2.44 | 4.20 | 1.00 | 4.20 | |
| 56 | 992917.1 | ferritin, heavy polypeptide 1 | 1.00 | 1.02 | 1.04 | 1.18 | 1.70 | 1.96 | 4.90 | 4.90 | 1.00 | 4.90 | |
| 57 | 411424.12 | LIM and senescent cell antigen-like domains 1 | 1.00 | 1.03 | 1.16 | 1.65 | 1.25 | 1.61 | 4.11 | 4.11 | 1.00 | 4.11 | |
| 58 | 995600.17 | Homo sapiens clone 24649 mRNA sequence | 1.00 | 1.00 | 0.99 | 1.07 | 1.04 | 1.03 | 5.85 | 5.85 | 0.99 | 5.85 | |
| 59 | 441292.7 | epithelial membrane protein 1 | 1.00 | 1.18 | 1.05 | 1.30 | 1.50 | 3.13 | 6.00 | 6.00 | 1.00 | 6.00 | |
| 60 | 42176.5 | Down syndrome candidate region 1 | 1.00 | 1.34 | 1.53 | 1.85 | 1.17 | 2.49 | 6.83 | 6.83 | 1.00 | 6.83 | |
| 61 | 234537.3 | 5′ nucleotidase (CD73) | 1.00 | 1.00 | 1.08 | 1.00 | 1.69 | 2.36 | 6.22 | 6.22 | 1.00 | 6.22 | |
| 62 | 470468.21 | uridine phosphorylase | 1.00 | 1.30 | 1.03 | 1.18 | 1.93 | 2.46 | 5.37 | 5.37 | 1.00 | 5.37 | |
| 63 | 240120.3 | diphtheria toxin receptor (heparin-binding | 1.00 | 1.03 | 0.95 | 0.98 | 1.82 | 2.95 | 7.00 | 7.00 | 0.95 | 7.00 | |
| | | epidermal growth factor-like growth fact | | | | | | | | | | | |
| 64 | 28779.3 | small inducible cytokine subfamily A | 1.00 | 0.97 | 1.00 | 1.26 | 0.56 | 2.02 | 8.18 | 8.18 | 0.56 | 8.18 | |
| | | (Cys-Cys), member 20 | | | | | | | | | | | |
| 65 | 238627.2 | BCL2-related protein A1 | 1.00 | 1.12 | 0.97 | 1.25 | 0.42 | 1.69 | 3.85 | 3.85 | 0.42 | 3.85 | |
| 66 | 254107.1 | thrombomodulin | 1.00 | 1.05 | 0.93 | 0.89 | 2.12 | 6.24 | 2.12 | 6.24 | 0.89 | 6.24 | |
| 67 | 330908.2 | leukemia inhibitory factor (cholinergic | 1.00 | 1.08 | 1.06 | 0.79 | 1.40 | 3.87 | 1.04 | 3.87 | 0.79 | 3.87 | |
| | | differentiation factor) | | | | | | | | | | | |
| 194 | 199898.3 | Human G0S2 protein gene, complete cds | 1.00 | 1.07 | 1.10 | 0.87 | 0.21 | 0.41 | 0.56 | 1.10 | 0.21 | 4.84 | down- |
| | | | | | | | | | | | | | regu- |
| | | | | | | | | | | | | | lated |
| 195 | 253550.14 | insulin-like growth factor binding protein 3 | 1.00 | 1.06 | 1.06 | 1.37 | 0.28 | 0.90 | 1.18 | 1.37 | 0.28 | 3.62 | |
| 196 | 331597.2 | cytochrome b-245, beta polypeptide | 1.00 | 1.14 | 1.08 | 1.02 | 0.25 | 0.28 | 0.16 | 1.14 | 0.16 | 6.32 | |
| | | (chronic granulomatous disease) | | | | | | | | | | | |
| 197 | 997377.1 | ribonuclease, RNase A family, 3 | 1.00 | 0.99 | 0.99 | 0.75 | 1.25 | 0.77 | 0.28 | 1.25 | 0.28 | 3.55 | |
| | | (eosinophil cationic protein) | | | | | | | | | | | |
| 198 | 42869.3 | cathepsin G | 1.00 | 1.14 | 1.04 | 0.80 | 1.60 | 0.84 | 0.20 | 1.60 | 0.20 | 4.98 | |
| 199 | 248306.1 | carbonic anhydrase II | 1.00 | 0.92 | 1.03 | 0.92 | 1.46 | 1.19 | 0.13 | 1.46 | 0.13 | 7.58 | |
| 200 | 247220.15 | thymidylate synthetase | 1.00 | 1.14 | 1.12 | 1.12 | 0.77 | 0.23 | 0.25 | 1.14 | 0.23 | 4.34 | |
| 201 | 26662.3 | centromere protein F (350/400 kD, mitosin) | 1.00 | 1.24 | 1.15 | 1.03 | 0.87 | 0.38 | 0.29 | 1.24 | 0.29 | 3.40 | |
| 202 | 977509.3 | v-myb avian myeloblastosis viral oncogene | 1.00 | 1.21 | 1.10 | 0.81 | 0.77 | 0.25 | 0.27 | 1.21 | 0.25 | 3.95 | |
| | | homolog-like 2 | | | | | | | | | | | |
| 203 | 221961.2 | myeloid cell nuclear differentiation antigen | 1.00 | 1.04 | 1.22 | 0.82 | 0.99 | 0.95 | 0.18 | 1.22 | 0.18 | 5.58 | |
| 204 | 246824.1 | ribonuclease, RNase A family, 2 | 1.00 | 1.15 | 1.03 | 0.97 | 1.17 | 0.71 | 0.19 | 1.17 | 0.19 | 5.33 | |
| | | (liver, eosinophil-derived neurotoxin) | | | | | | | | | | | |
| 205 | 407557.2 | cyclin-dependent kinase inhibitor | 1.00 | 1.21 | 1.03 | 0.80 | 1.09 | 0.43 | 0.24 | 1.21 | 0.24 | 4.17 | |
| | | 2C (p18, inhibits CDK4) | | | | | | | | | | | |
| 206 | 372981.2 | Homo sapiens ZW10 interactor | 1.00 | 1.10 | 1.07 | 1.17 | 0.62 | 0.30 | 0.29 | 1.17 | 0.29 | 3.48 | |
| | | Zwint mRNA, complete cds | | | | | | | | | | | |
| 207 | 201409.6 | Fc fragment of IgG, high affinity | 1.00 | 1.25 | 1.07 | 1.21 | 0.90 | 0.58 | 0.23 | 1.25 | 0.23 | 4.27 | |
| | | Ia, receptor for (CD64) | | | | | | | | | | | |
| 208 | 331025.1 | Homo sapiens mitotic centromere-associated | 1.00 | 1.10 | 1.04 | 0.91 | 0.90 | 0.48 | 0.26 | 1.10 | 0.26 | 3.89 | |
| | | kinesin mRNA, complete cds | | | | | | | | | | | |
| 209 | 247515.1 | elastase 2, neutrophil | 1.00 | 0.96 | 1.11 | 0.95 | 1.11 | 1.08 | 0.29 | 1.11 | 0.29 | 3.48 | |
| 210 | 199471.2 | MAD2 (mitotic arrest deficient, | 1.00 | 1.05 | 1.02 | 0.69 | 0.99 | 0.28 | 0.31 | 1.05 | 0.28 | 3.60 | |
| | | yeast, homolog)-like 1 | | | | | | | | | | | |
| 211 | 2916753 | high-mobility group (nonhistone | 1.00 | 1.05 | 0.99 | 1.25 | 0.94 | 0.36 | 0.19 | 1.25 | 0.19 | 5.37 | |
| | | chromosomal) protein 2 | | | | | | | | | | | |
| 212 | 343899.2 | hyaluronan-mediated motility | 1.00 | 1.08 | 1.14 | 0.94 | 0.88 | 1.02 | 0.26 | 1.14 | 0.26 | 3.81 | |
| | | receptor (RHAMM) | | | | | | | | | | | |
| 213 | 335775.2 | lamin B1 | 1.00 | 1.05 | 1.09 | 1.07 | 0.52 | 0.32 | 0.18 | 1.09 | 0.18 | 5.44 | |

What is claimed is:

1. A composition comprising a plurality of polynucleotides that are differentially expressed in foam cell development and selected from SEQ ID NOs:1-276 or a complement thereof.

2. The composition of claim 1, wherein each of the polynucleotides is differentially expressed early in foam cell development and is selected from
(a) SEQ ID NOs:1-55;
(b) SEQ ID NOs:171-196; or
(c) a complement of (a) or (b).

3. The composition of claim 1, wherein each of the polynucleotides is differentially expressed greater than 3-fold and is selected from
(a) SEQ ID NOs:47-67;
(b) SEQ ID NOs:194-213; or
(c) a complement of (a) or (b).

4. The composition of claim 1, wherein the polynucleotides are immobilized on a substrate.

5. A high throughput method for detecting altered expression of one or more polynucleotides in a sample, the method comprising:
(a) hybridizing the composition of claim 2 with the sample, thereby forming one or more hybridization complexes;
(b) detecting the hybridization complexes; and
(c) comparing the hybridization complexes with those of a standard, wherein each difference in the size and intensity of a hybridization complex indicates altered expression of a polynucleotide in the sample.

6. The method of claim 5, wherein the sample is from a subject with atherosclerosis and comparison with a standard defines early, mid, and late stages of that disease.

7. A high throughput method of screening a library of molecules or compounds to identify a ligand which binds a polynucleotide, the method comprising:
(a) combining the composition of claim 1 with the library under conditions to allow specific binding; and
(b) detecting specific binding between the polynucleotide and a molecule or compound, thereby identifying a ligand that specifically binds to the polynucleotide.

8. The method of claim 7 wherein the library is selected from DNA molecules, RNA molecules, peptide nucleic acids, mimetics, peptides, and proteins.

9. A method of obtaining an extended or full length gene from a library of nucleic acid sequences, the method comprising:
(a) arranging individual sequences on a substrate;
(a) hybridizing a polynucleotide selected from claim 1 with the sequences under conditions which allow specific binding;
(b) detecting hybridization between the polynucleotide and one or more sequences; and
(c) isolating the sequences from the library, thereby obtaining extended or full length gene.

10. A substantially purified polynucleotide selected from SEQ ID NOs:35-48, 68-80, 192, 193, and 214-222.

11. An expression vector containing the polynucleotide of claim 10.

12. A host cell containing the expression vector of claim 11.

13. A method for producing a protein, the method comprising the steps of:
(a) culturing the host cell of claim 12 under conditions for the expression of protein; and
(b) recovering the protein from the host cell culture.

14. A protein produced by the method of claim 13.

15. A high-throughput method for screening a library of molecules or compounds to identify at least one ligand which specifically binds a protein, the method comprising:
(a) combining the protein or a portion thereof of claim 14 with the library under conditions to allow specific binding; and
(b) detecting specific binding between the protein and a molecule or compound, thereby identifying a ligand which specifically binds the protein.

16. The method of claim 15 wherein the library is selected from DNA molecules, RNA molecules, PNAs, mimetics, peptides, proteins, agonists, antagonists, antibodies or their fragments, immunoglobulins, inhibitors, drug compounds, and pharmaceutical agents.

17. A method of purifying a ligand from a sample, the method comprising:
a) combining the protein of claim 15 with a sample under conditions to allow specific binding;
b) recovering the bound protein; and
c) separating the protein from the ligand, thereby obtaining purified ligand.

18. A pharmaceutical composition comprising the protein of claim 14 in conjunction with a pharmaceutical carrier.

19. A purified antibody that specifically binds to the protein of claim 14.
